SEQUENCE LISTING (63 sequences)

SEQ ID NO: 1 - protein, 21 aa, Homo sapiens
EPKSCDKTHTCPPCPAPELLG

SEQ ID NO: 2 - protein, 19 aa, Homo sapiens
ERKCCVECPPCPAPPVAGP

SEQ ID NO: 3 - protein, 53 aa, Homo sapiens
ELKTPLGDTTHTCPRCPEPKSCDTPPPCPRCPEPKSCDTPPPCPRCPAPELLG

SEQ ID NO: 4 - protein, 18 aa, Homo sapiens
ESKYGPPCPSCPAPEFLG

SEQ ID NO: 5 - protein, 103 aa, Homo sapiens
APPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEK

SEQ ID NO: 6 - protein, 103 aa, Homo sapiens
APPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEK

SEQ ID NO: 7 - protein, 9 aa, Homo sapiens
SLYNTVATL

SEQ ID NO: 8 - protein, 480 aa, Homo sapiens
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGSGGGGEAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNGEPKSSDKTHTCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 461 aa, Homo sapiens
QKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHIGGGSGGGGDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGEPKSSDKTHTCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 10 - protein, 478 aa, Homo sapiens
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGSGGGGEAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 459 aa, Homo sapiens
QKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHIGGGSGGGGDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 12 - protein, 478 aa, Homo sapiens
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGSGGGGEAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 459 aa, Homo sapiens
QKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHIGGGSGGGGDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 14 - protein, 478 aa, Homo sapiens
EVQLVESGGGLVQPGGSLRLSCAASGYSFTGYTMNWVRQAPGKCLEWVALINPYKGVSTYNQKFKDRFTISVDKSKNTAYLQMNSLRAEDTAVYYCARSGYYGDSDWYFDVWGQGTLVTVSSGGGSGGGGEAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 459 aa, Homo sapiens
QKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHIGGGSGGGGDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGCGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 16 - protein, 474 aa, Homo sapiens
QKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHIGGGSGGGGEVQLVESGGGLVQPGGSLRLSCAASGYSFTGYTMNWVRQAPGKCLEWVALINPYKGVSTYNQKFKDRFTISVDKSKNTAYLQMNSLRAEDTAVYYCARSGYYGDSDWYFDVWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 17 - protein, 463 aa, Homo sapiens
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGCGTKVEIKGGGSGGGGEAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 462 aa, Homo sapiens
EAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNGGGSGGGGQIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 472 aa, Homo sapiens
EVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYDGFVYWGQGTLVTVSSGGGSGGGGQKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHIEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 20 - protein, 467 aa, Homo sapiens
EAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNGGGGSGGGGSGGGQIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 472 aa, Homo sapiens; identical to SEQ ID NO: 19

SEQ ID NO: 22 - protein, 462 aa, Homo sapiens
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGEAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 472 aa, Homo sapiens
QKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHIGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYDGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 24 - protein, 468 aa, Homo sapiens
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGGSGGGGSGGGGEAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 472 aa, Homo sapiens; identical to SEQ ID NO: 23

SEQ ID NO: 26 - protein, 231 aa, Homo sapiens
EPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 27 - protein, 231 aa, Homo sapiens
EPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 113 aa, Homo sapiens
QKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHI

SEQ ID NO: 29 - protein, 122 aa, Homo sapiens
EVQLVESGGGLVQPGGSLRLSCAASGYSFTGYTMNWVRQAPGKCLEWVALINPYKGVSTYNQKFKDRFTISVDKSKNTAYLQMNSLRAEDTAVYYCARSGYYGDSDWYFDVWGQGTLVTVSS

SEQ ID NO: 30 - protein, 8 aa, Homo sapiens
GGGSGGGG

SEQ ID NO: 31 - protein, 117 aa, Homo sapiens
EAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKN

SEQ ID NO: 32 - protein, 107 aa, Homo sapiens
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGCGTKVEIK

SEQ ID NO: 33 - protein, 478 aa, Homo sapiens
EAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNGSADDAKKDAAKKDGKSQKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHIEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 34 - protein, 451 aa, Homo sapiens
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 698 aa, Homo sapiens
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKEAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNGSADDAKKDAAKKDGKSQKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHI

SEQ ID NO: 36 - protein, 692 aa, Homo sapiens
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKEAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNGSADDAKKDAAKKDGKSQKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHI

SEQ ID NO: 37 - protein, 698 aa, Homo sapiens
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNGSADDAKKDAAKKDGKSQKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHI

SEQ ID NO: 38 - protein, 696 aa, Homo sapiens
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGKEAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNGSADDAKKDAAKKDGKSQKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHI

SEQ ID NO: 39 - protein, 690 aa, Homo sapiens
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGECESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKEAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNGSADDAKKDAAKKDGKSQKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHI

SEQ ID NO: 40 - protein, 428 aa, Homo sapiens
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGECDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 41 - protein, 1392 aa, Homo sapiens
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKEAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNGSADDAKKDAAKKDGKSQKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHIEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKEAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNGSADDAKKDAAKKDGKSQKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHI

SEQ ID NO: 42 - protein, 214 aa, Homo sapiens
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 461 aa, Homo sapiens
QKEVEQNSGPLSVPEGAIASLNCTYSDRGSQSFFWYRQYSGKSPELIMSIYSNGDKEDGRFTAQLNKASQYFSLLIRDSQPSDSATYLCAAVIDNSNGGILTFGTGTRLTIIPNIQNGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 44 - protein, 477 aa, Homo sapiens
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGKAGVTQTPRYLIKTRGQQVTLSCSPIPGHRSVSWYQQTPGQGLQFLFEYVHGAERNKGNFPGRFSGRQFSNSSSEMNISNLELGDSALYLCASSPWDSPNEQYFGPGTRLTVTEDLKNEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 45 - protein, 463 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAAVIDNDQGGILTFGTGTRLTIIPNIQNGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 479 aa, Homo sapiens
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGKAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKNEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 47 - protein, 229 aa, Homo sapiens
EPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 48 - protein, 229 aa, Homo sapiens
EPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 49 - protein, 9 aa, Homo sapiens
SLLQHLIGL

SEQ ID NO: 50 - protein, 6 aa, Homo sapiens
ELLGGP

SEQ ID NO: 51 - protein, 457 aa, Homo sapiens
QKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHIGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 52 - protein, 476 aa, Homo sapiens
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGEAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 53 - protein, 457 aa, Homo sapiens
DIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKGGGSGGGGQKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHIEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 54 - protein, 476 aa, Homo sapiens
EAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNGGGSGGGGEVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 55 - protein, 461 aa, Homo sapiens
EAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 56 - protein, 472 aa, Homo sapiens
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGQKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHIEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 57 - protein, 462 aa, Homo sapiens
DIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKGGGSGGGGGEAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 58 - protein, 472 aa, Homo sapiens
QKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHIGGGSGGGGEVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 59 - protein, 4 aa, synthetic construct
  REGION 1..4: note = Linker
GGGS

SEQ ID NO: 60 - protein, 5 aa, synthetic construct
  REGION 1..5: note = Linker
GGGGS

SEQ ID NO: 61 - protein, 5 aa, synthetic construct
  REGION 1..5: note = Linker
TVLRT

SEQ ID NO: 62 - protein, 6 aa, synthetic construct
  REGION 1..6: note = Linker
TVSSAS

SEQ ID NO: 63 - protein, 7 aa, synthetic construct
  REGION 1..7: note = Linker
TVLSSAS

(19) United States
(12) Patent Application Publication
(10) Pub. No.: US 2025/0361300 A1
(43) Pub. Date: Nov. 27, 2025

(54) IMPROVED DUAL SPECIFICITY POLYPEPTIDE MOLECULE

(71) Applicant: Immatics Biotechnologies GmbH, Tübingen (DE)

(72) Inventors: Martin HOFMANN, Tübingen (DE); Felix UNVERDORBEN, Tübingen (DE); Sebastian BUNK, Tübingen (DE); Dominik MAURER, Tübingen (DE)

(21) Appl. No.: 19/240,392

(22) Filed: Jun. 17, 2025

Related U.S. Application Data
(63) Continuation of application No. 18/331,556, filed on Jun. 8, 2023.
Application No. 18/331,556 is a continuation of application No. 16/035,403, filed on Jul. 13, 2018, now Pat. No. 11,905,328.
(60) Provisional application No. 62/532,713, filed on Jul. 14, 2017.
(60) Provisional application No. 62/658,318, filed on Apr. 16, 2018.

(30) Foreign Application Priority Data
Jul. 14, 2017 (DE) 102017115966.5
Aug. 30, 2017 (DE) 102017119866.0
Apr. 16, 2018 (DE) 102018108995.3

(51) Int. Cl. C07K 16/28; C07K 16/10; C07K 16/30; C07K 16/46

(52) U.S. Cl. CPC C07K 16/2809; C07K 16/1045; C07K 16/2833; C07K 16/30; C07K 16/46; C07K 16/468; C07K 2317/24; C07K 2317/31; C07K 2317/32; C07K 2317/626; C07K 2317/94

(57) ABSTRACT

The present invention relates to a bispecific polypeptide molecule comprising a first polypeptide chain and a second polypeptide chain providing a binding region derived from a T cell receptor (TCR) being specific for a major histocompatibility complex (MHC)-associated peptide epitope, and a binding region derived from an antibody capable of recruiting human immune effector cells by specifically binding to a surface antigen of said cells, as well as methods of making the bispecific polypeptide molecule, and uses thereof.

### CROSS REFERENCE TO RELATED APPLICATIONS

This application is a continuation of U.S. patent application Ser. No. 18/331,556, filed 8 Jun. 2023, which is a continuation of U.S. patent application Ser. No. 16/035,403, filed 13 Jul. 2018, now U.S. Pat. No. 11,905,328, issued 20 Feb. 2024, which claims the benefit of U.S. Provisional Application No. 62/658,318, filed 16 Apr. 2018, U.S. Provisional Application No. 62/532,713, filed 14 Jul. 2017, German Application No. 102018108995.3, filed 16 Apr. 2018, German Application No. 102017119866.0, filed 30 Aug. 2017, and German Application No. 102017115966.5 filed 14 Jul. 2017. The entire contents of each of these listed applications are incorporated herein by reference for all purposes.

This application is also related to PCT/EP2018/069151 and PCT/EP2018/069157, both filed on 13 Jul. 2018. The entire contents of each of these listed applications are incorporated herein by reference for all purposes.

### REFERENCE TO SEQUENCE LISTING SUBMITTED AS A COMPLIANT ASCII TEXT FILE (.xml)

Pursuant to the EFS-Web legal framework and 37 CFR §§ 1.821-825 (see MPEP § 2442.03 (a)), a Sequence Listing in the form of an xml file (entitled “3000058-009009_Sequence_Listing.xml” created on 16 Jun. 2025 and 78,811 bytes in size) is submitted concurrently with the instant application, and the entire contents of the Sequence Listing are incorporated herein by reference.

### FIELD

The present invention relates to a bispecific polypeptide molecule comprising a first polypeptide chain and a second polypeptide chain providing a binding region derived from a T cell receptor (TCR) being specific for a major histocompatibility complex (MHC)-associated peptide epitope, and a binding region derived from an antibody capable of recruiting human immune effector cells by specifically binding to a surface antigen of said cells, as well as methods of making the bispecific polypeptide molecule, and uses thereof.

### BACKGROUND OF THE INVENTION

With the development of molecular cloning technology and the deep understanding of antibody engineering, there are diverse bispecific antibody formats (“bispecifics”) from which to choose in order to achieve the optimal biological activity and clinical purpose. In cancer therapy, bispecific antibodies have been developed with the purpose of redirecting the activity of immune effector cells to the site of tumor through a first binding domain specific for an epitope on tumor cells and a second binding domain specific for an epitope on the immune effector cells. Bispecific antibodies for retargeting of immune effector cells have been developed in different formats, including formats without fragment crystallizable (Fc) region and IgG-derived formats with symmetric or asymmetric design. Besides retargeting effector cells to the site of cancer, new applications were established for bispecific antibodies. Bispecifics that can inhibit two correlated signaling molecules at the same time can be developed to overcome inherent or acquired resistance and to be more efficient angiogenesis inhibitors. In addition, bispecific antibodies can be employed as promising immune-stimulatory agents to treat various diseases like cancer. Bispecific antibodies can also be used to treat hemophilia A by mimicking the function of factor VIII. Bispecific antibodies also have broad application prospects in bone disorders and infections and diseases of the central nervous system (reviewed in Yang F. et al. Bispecific Antibodies as a Development Platform for New Concepts and Treatment Strategies. Int J Mol Sci. 2016 Dec. 28; 18 (1)).

T cells express T cell receptor (TCR) complexes that are able to induce antigen-specific immune responses. Engagement of antigen peptide/major histocompatibility complex (MHC) Class I on the target cell with the TCR induces the formation of an immune synapse and leads to signaling through CD3 co-receptors, which are components of the TCR signaling complex. This signaling cascade directs T cell-mediated killing of the cell expressing the antigen through the release and transfer of granzymes and perforin from the T cell to the target cell.

Historically, discovery and production of single-chain connected variable domains of antibodies (scFvs, described by Bird et al. 1988) served as major driver for the development of bispecific antibodies. This concept finally led to generation of BiTE-molecules and their clinical validation as a potent drug for the treatment of leukemia (Baeuerle, P. A.; Reinhardt, C. Bispecific T-cell engaging antibodies for cancer therapy. Cancer Res. 2009, 69, 4941-4944). In cancer, bispecific antibodies that co-engage the CD3 epsilon subunit and a surface antigen on the tumor cell trigger T cell-mediated killing of the tumor cell while circumventing the need for the direct interaction of the TCR and MHC class I in complex with antigen. This expands the repertoire of T cells able to recognize the tumor and act as effector cells (Baeuerle, P. A.; Reinhardt, C. Bispecific T-cell engaging antibodies for cancer therapy. Cancer Res. 2009, 69, 4941-4944).

Stieglmaier J., et al. (in: Utilizing the BiTE (bispecific T-cell engager) platform for immunotherapy of cancer. Expert Opin Biol Ther. 2015; 15 (8): 1093-9) describe that various approaches of T-cell-based cancer immunotherapy are currently under investigation, among these are BiTE® (bispecific T-cell engager) antibody constructs, which have a unique design and mechanism of action. They are constructed by genetically linking onto a single polypeptide chain the minimal binding domains of monoclonal antibodies for tumor-associated surface antigens and for the T-cell receptor-associated molecule CD3. Concurrent engagement of the target cell antigen and CD3 leads to activation of polyclonal cytotoxic T-cells, resulting in target cell lysis. Blinatumomab, a BiTE® targeting CD19, is being investigated in a broad range of B-cell malignancies and has recently been approved in the USA by the US FDA for Philadelphia chromosome-negative relapsed/refractory B-acute lymphoblastic leukemia under the trade name BLINCYTO™. The BiTE® platform is one of the clinically most advanced T-cell immunotherapy options.

However the shortcomings of small bispecific molecules, like BiTEs®, have been discovered to be poor production yields, difficult purification processes, aggregation propensity and also a very short serum half-life. To overcome the inherent limitations of this class of molecules various bispecific formats based on human IgG were developed starting with the concept of recombinant bispecific prototype immunoglobulin (Ig)-G-like antibodies as devised more than two decades ago, when Morrison and colleagues fused flexible linker peptides to the C termini of the heavy chains of IgG followed by single-chain variable domains with different binding specificities (Coloma, M. J. and Morrison, S. L. (1997) Design and production of novel tetravalent bispecific antibodies. Nat. Biotechnol. 15, 159-163). The molecules could be differentiated from ‘normal’ antibodies because they had dual functionalities. Technical hurdles initially hampered further development, causing bispecific antibodies (bsAbs) to remain a topic of R&D primarily in the academic and biotech environment. However, rapidly evolving technologies that enabled the engineering, production, and development of recombinant protein derivatives, combined with renewed interest from the pharmaceutical industry, jump-started the bsAb research field. Today, many different bsAb formats suitable for the development of therapeutic proteins are available (for reviews, see Gramer, mAbs. 2013; 5 (6): 962-973, Weidle, Cancer Genomics Proteomics. 2013 November-December; 10 (6): 239-50, Brinkmann, MAbs. 2017 February/March; 9 (2): 182-212.). In summary, the inclusion of Fc-(fragment crystalizable) parts, consisting of CH2 and CH3 domains led to increased productivity, simplified purification processes and enhanced stability. In addition the serum half-life of such IgG-based drugs was prolonged due to i) the increase in size and ii) the interaction of the Fc-part with the human Fc-receptor FcRn.

Development of IgG-based bispecific formats was further fueled by the advent and incorporation of engineered mutations to facilitate the hetero-dimerization of two differing CH3-domains thereby connecting two different polypeptide chains. The basic concept was introduced by Ridgway J B, et al. (in: ‘Knobs-into-holes’ engineering of antibody CH3 domains for heavy chain heterodimerization. Protein Eng. 1996 July; 9 (7): 617-21) who disclosed the ‘knobs-into-holes’ approach as a novel and effective design strategy for engineering antibody heavy chain homodimers for heterodimerization. In this approach a ‘knob’ variant was first obtained by replacement of a small amino acid with a larger one in the CH3 domain of a CD4-IgG immunoadhesin: T366Y. The knob was designed to insert into a ‘hole’ in the CH3 domain of a humanized anti-CD3 antibody created by judicious replacement of a large residue with a smaller one: Y407T. The anti-CD3/CD4-IgG hybrid represents up to 92% of the protein A purified protein pool following co-expression of these two different heavy chains together with the anti-CD3 light chain. In contrast, only up to 57% of the anti-CD3/CD4-IgG hybrid is recovered following co-expression in which heavy chains contained wild-type CH3 domains. Thus knobs-into-holes engineering facilitates the construction of an antibody/immunoadhesin hybrid and likely other Fc-containing bifunctional therapeutics including bispecific immunoadhesins and bispecific antibodies. Atwell et al, 1997, J Mol Biol (Stable heterodimers from remodeling the domain interface of a homodimer using a phage display library) discloses a knob-into-hole mutation (knob: T366W/hole: T366S+L368A+Y407V) in the CH3 domain of the Fc domain for improved heterodimerization. This concept was further improved by the additional introduction of cysteine-residues to form a stabilizing disulfide-bond between the heterodimeric CH3-domains as described by Merchant et al. 1998, Nature Biotechnology (An Efficient Route to Human Bispecific IgG).

Further concepts to produce heterodimeric molecules were disclosed by Muda et al. 2011, PEDS (Therapeutic assessment of SEED: a new engineered antibody platform designed to generate mono- and bispecific antibodies); Gunasekaran et al. 2010, J Biol Chem (Enhancing antibody Fc heterodimer formation through electrostatic steering effects: applications to bispecific molecules and monovalent IgG); Moore et al. 2011, MAbs (A novel bispecific antibody format enables simultaneous bivalent and monovalent co-engagement of distinct target antigens); Von Kreudenstein et al. 2013, MAbs (Improving biophysical properties of a bispecific antibody scaffold to aid developability: quality by molecular design.) These concepts are summarized and reviewed by Ha et al. 2016, Front Immunol (Immunoglobulin Fc Heterodimer Platform Technology: From Design to Application in Therapeutic Antibodies and Proteins) and Liu et al. 2017, Front Immunol (Fc Engineering for Developing Therapeutic Bispecific Antibodies and Novel scaffolds).

With the inclusion of Fc-parts consisting of Hinges, CH2 and CH3 domains, or parts thereof, into bispecific molecules the problem of unspecific immobilization of these molecules, induced by Fc: Fc-gamma receptor (FcgR) interactions arose. FcgRs are composed of different cell surface molecules (FcgRI, FcgRIIa, FcgRIIb, FcgRIII) binding with differing affinities to epitopes displayed by Fc-parts of IgG-molecules. As such an unspecific (i.e. not induced by either of the two binding domains of an bispecific molecule) immobilization is unfavorable due to i) influence on pharmacokinetics of a molecule and ii) off-target activation of immune effector cells various Fc-variants and mutations to ablate FcgR-binding have been identified.

Morgan et al. 1995, Immunology (The N-terminal end of the CH2 domain of chimeric human IgG1 anti-HLA-DR is necessary for C1q, FcγRI and FcγRIII binding) disclose the exchange of the residues 233-236 of human IgG1 with the corresponding sequence derived from human IgG2 resulting in abolished FcgRI binding, abolished C1q binding and diminished FcgRIII binding.

EP1075496 discloses antibodies and other Fc-containing molecules with variations in the Fc region (233P, 234V, 235A and no residue or G in position 236 and 327G, 330S and 331S) wherein the recombinant antibody is capable of binding the target molecule without triggering significant complement dependent lysis, or cell mediated destruction of the target.

Dual affinity retargeting (DART) molecules are used in order to achieve, for example, an optimal redirected T-cell killing of B-cell lymphoma. The original DART technology is described in Moore et al. (in: Application of dual affinity retargeting molecules to achieve optimal redirected T-cell killing of B-cell lymphoma, Blood. 2011 Apr. 28; 117 (17): 4542-51). Comparison with a single-chain, bispecific antibody bearing identical CD19 and CD3 antibody Fv sequences revealed DART molecules to be more potent in directing B-cell lysis. Further evolution of the DART technology was achieved by the DART-Fc-molecules as described in Root et al, 2016 antibodies (Development of PF-06671008, a Highly Potent Anti-P-cadherin/Anti-CD3 Bispecific DART Molecule with Extended Half-Life for the Treatment of Cancer). This molecule combined the high potency of the DARTs with, among other positive characteristics, the extended serum half-life of Fc-based molecules.

The αβTCR (TCR) recognizes antigenic peptides presented by MHC and is responsible for the specificity of T cells. Both a and B chains of the TCR possess variable (V) and constant domains. The V domains are involved in binding antigenic peptide and the constant domains traverse through the T cell membrane. From crystal structure analysis of TCR bound to peptide-MHC complex, complementarity determining regions (CDR) 3 of both the V_{α} and V_{β} chains preferably interact with peptide, while CDRs 1 and 2 interact with MHC. However, recognition of peptide by CDR 1 and recognition of MHC by CDR 3 has also been described (Piepenbrink et al, The basis for limited specificity and MHC restriction in a T cell receptor interface, Nat Commun, 2013; 4, 1948). The TCR αß heterodimer is closely associated with CD3 proteins, CD4 or CD8, and other adhesion and signal transducing proteins. Binding of antigenic peptide by the TCR V regions triggers T cell activation by signal transduction through the TCR constant domains via CD3 and CD4 or CD8 cytoplasmic proteins.

Single-chain TCRs (scTCRs) afford significant advantages in contrast to the full-length TCR format for engineering, soluble protein expression, and clinical potential. From the perspective of soluble protein expression (i.e. manufacturing), scTCRs are produced as a single polypeptide, avoiding the requirement for production of each TCR chain as separate polypeptides and allowing for production of larger quantities of the properly assembled scTCR that binds to its peptide-MHC ligand. This feature can allow for production yields that are necessary for clinical use. Finally, from the clinical perspective, scTCRs consisting of only the V regions (scTv) can be formatted as therapeutics or diagnostic reagents similar to scFv fragments.

US 2006-0166875 discloses a single chain T cell receptor (scTCR) comprising a segment constituted by a TCR alpha chain variable region sequence fused to the N terminus of a TCR alpha chain constant region extracellular sequence, a beta segment constituted by a TCR beta chain variable region fused to the N terminus of a TCR beta chain constant region extracellular sequence, and a linker sequence linking the C terminus of the a segment to the N terminus of the beta segment, or vice versa, the constant region extracellular sequences of the alpha and beta segments being linked by a disulfide bond, the length of the linker sequence and the position of the disulfide bond being such that the variable region sequences of the alpha and beta segments are mutually orientated substantially as in native alpha/beta T cell receptors. Complexes of two or more such scTCRs, and use of the scTCRs in therapy and in various screening applications are also disclosed. In contrast to the scTCR described in US 2006-0166875, US 2012-0252742 discloses a soluble human single chain TCR without constant domains, consisting of only the variable fragments of the TCR (scTv), which is useful for many purposes, including the treatment of cancer, viral diseases and autoimmune diseases.

McCormack E, et al (in: Bi-specific TCR-anti CD3 redirected T-cell targeting of NY-ESO-1- and LAGE-1-positive tumors. Cancer Immunol Immunother. 2013 April; 62 (4): 773-85) disclose that NY-ESO-1 and LAGE-1 are cancer testis antigens with an ideal profile for tumor immunotherapy, combining up-regulation in many cancer types with highly restricted expression in normal tissues and sharing a common HLA-A*0201 epitope, 157-165. They present data to describe the specificity and anti-tumor activity of a bifunctional ImmTAC, comprising a soluble, high-affinity T-cell receptor (TCR) specific for NY-ESO-1157-165 fused to an anti-CD3 scFv. This reagent, ImmTAC-NYE, is shown to kill HLA-A2, antigen-positive tumor cell lines, and freshly isolated HLA-A2- and LAGE-1-positive NSCLC cells. Employing in vivo optical imaging, the results show in vivo targeting of fluorescently labelled high-affinity NYESO-specific TCRs to HLA-A2-, NY-ESO-1157-165-positive tumors in xenografted mice. In vivo ImmTAC-NYE efficacy was tested in a tumor model in which human lymphocytes were stably co-engrafted into immunodeficient NSG mice harboring tumor xenografts; efficacy was observed in both tumor prevention and established tumor models using a GFP fluorescence readout. Quantitative RT-PCR was used to analyze the expression of both NY-ESO-1 and LAGE-1 antigens in 15 normal tissues, 5 cancer cell lines, 10 NSCLC, and 10 ovarian cancer samples. Overall, LAGE-1 RNA was expressed at a greater frequency and at higher levels than NY-ESO-1 in the tumor samples. ImmTACs comprise a single-chain Fv derived from anti-CD3 antibody UCHT-1 covalently linked to the C- or N-terminus of the alpha or beta chain of the TCR.

EP1868650 is directed at diabody molecules and uses thereof in the treatment of a variety of diseases and disorders, including immunological disorders, infectious disease, intoxication and cancers. The diabody molecules comprise two polypeptide chains that associate to form at least two epitope binding sites, which may recognize the same or different epitopes on the same or differing antigens. Additionally, the antigens may be from the same or different molecules. The individual polypeptide chains of the diabody molecule may be covalently bound through non-peptide bond covalent bonds, such as, but not limited to, disulfide bonding of cysteine residues located within each polypeptide chain. In particular embodiments, the diabody molecules further comprise an Fc region, which is disclosed herein as it allows engineering of antibody-like properties (e.g. long half-life) into the molecule. EP1868650 requires the presence of binding regions of light chain or heavy chain variable domains of an immunoglobulin, and extensively discusses functional Fc receptor binders.

WO 2016/184592 A1 discloses bispecific molecules in which one specificity is contributed by a TCR and the other by an antibody, which is directed against an antigen or epitope on the surface of lymphocytes, but does not disclose the specific arrangement of the elements of the TCR and the antibody variable regions as disclosed herein.

EP2258720A1 is directed to a functional T cell receptor (TCR) fusion protein (TFP) recognizing and binding to at least one MHC-presented epitope, and containing at least one amino acid sequence recognizing and binding an antigen.

It is an object of the present invention to provide improved bispecific molecules capable of targeting peptide-MHC-complexes, that can be easily produced, display high stability and also provide high potency when binding to the respective antigen epitopes. Other objects and advantages of the present invention will become apparent when studying the following description and the preferred embodiments thereof, as well as the respective examples.

In a first aspect of the invention, the above object is solved by providing a dual specificity polypeptide molecule selected from the group of molecules comprising a first polypeptide chain and a second polypeptide chain, wherein:
the first polypeptide chain comprises a first binding region of a variable domain (VD1) of an antibody specifically binding to a cell surface antigen of a human immune effector cell, and
a first binding region of a variable domain (VR1) of a TCR specifically binding to an MHC-associated peptide epitope, and
a first linker (LINK1) connecting said domains;
the second polypeptide chain comprises a second binding region of a variable domain (VR2) of a TCR specifically binding to an MHC-associated peptide epitope, and
a second binding region of a variable domain (VD2) of an antibody specifically binding to a cell surface antigen of a human immune effector cell, and
a second linker (LINK2) connecting said domains;
wherein said first binding region (VD1) and said second binding region (VD2) associate to form a first binding site (VD1) (VD2) that binds the epitope of the cell surface molecule;
said first binding region (VR1) and said second binding region (VR2) associate to form a second binding site (VR1) (VR2) that binds said MHC-associated peptide epitope;
wherein said two polypeptide chains are fused to human IgG hinge domains and/or human IgG Fc domains or dimerizing portions thereof; and
wherein the said two polypeptide chains are connected by covalent and/or non-covalent bonds between said hinge domains and/or Fc-domains; and
wherein said dual specificity polypeptide molecule is capable of simultaneously binding the cell surface molecule and the MHC-associated peptide epitope, and
dual specificity polypeptide molecules, wherein the order of the binding regions in the polypeptide chains is selected from VD1-VR1; VD1-VR2; VD2-VR1; VD2-VR2; VR1-VD1; VR1-VD2; VR2-VD1; VR2-VD2, and wherein the domains are either connected by LINK1 or LINK2. Linker sequences LINK1 and/or LINK2 contain at least one sequence motif selected from GGGS (SEQ ID NO: 59), GGGGS (SEQ ID NO: 60), TVLRT (SEQ ID NO: 61), TVSSAS (SEQ ID NO: 62), and TVLSSAS (SEQ ID NO: 63). Antibody-driven domains VD1 and VD2 display an engineered disulfide bridge introducing a covalent bond between VD1 and VD2 and where said cysteines are introduced into framework region (FR) 4 in case of VL and framework region 2 in case of VH.

Preferred is a dual specificity polypeptide molecule comprising a first polypeptide chain and a second polypeptide chain, wherein: the first polypeptide chain comprises a first binding region of a variable domain (VD1) derived from an antibody capable of recruiting human immune effector cells by specifically binding to a surface antigen of said cells, and a first binding region of a variable domain (VR1) derived from a TCR being specific for an MHC-associated peptide epitope, and a first linker portion (LINK1) connecting the two domains; the second polypeptide chain comprises a second binding region of a variable domain (VR2) derived from a TCR being specific for an MHC-associated peptide epitope, and a second binding region of a variable domain (VD2) derived from an antibody capable of recruiting human immune effector cells by specifically binding to a surface antigen of said cells, and a second linker portion (LINK2) connecting the two domains; wherein said first binding region (VD1) and said second binding region (VD2) associate to form a first binding site (VD1) (VD2) that binds the epitope of the cell surface molecule; said first binding region (VR1) and said second binding region (VR2) associate to form a second binding site (VR1) (VR2) that binds said MHC-associated peptide epitope; wherein at least one of said polypeptide chains is connected at its c-terminus to hinge-regions, CH2 and/or CH3-domains or parts thereof derived from human IgG; and wherein said dual specificity polypeptide molecule is capable of simultaneously binding the immune effector cell antigen and the MHC-associated peptide epitope.

Preferably, the dual specificity polypeptide molecule according to the present invention binds with high specificity to both the immune effector cell antigen and a specific antigen epitope presented as a peptide-MHC complex, e.g. with a binding affinity (KD) of about 100 nM or less, about 30 nM or less, about 10 nM or less, about 3 nM or less, about 1 nM or less, e.g. measured by Bio-Layer Interferometry as described in Example 6 or as determined by flow cytometry.

The inventive dual specificity polypeptide molecules according to the present invention are exemplified here by a dual specificity polypeptide molecule comprising a first polypeptide chain comprising SEQ ID No. 16 and a second polypeptide chain comprising SEQ ID No. 17.

In a second aspect of the invention, the above object is solved by providing a nucleic acid(s) encoding for a first polypeptide chain and/or a second polypeptide chain as disclosed herein, or expression vector(s) comprising such nucleic acid. In a third aspect of the invention, the above object is solved by providing a host cell comprising vector(s) as defined herein.

In a fourth aspect of the invention, the above object is solved by providing a method for producing a dual specificity polypeptide molecule according to the present invention, comprising suitable expression of said expression vector(s) comprising the nucleic acid(s) as disclosed in a suitable host cell, and suitable purification of the molecule(s) from the cell and/or the medium thereof.

In a fifth aspect of the invention, the above object is solved by providing a pharmaceutical composition comprising the dual specificity polypeptide molecule according to the invention, the nucleic acid or the expression vector(s) according to the invention, or the cell according to the invention, together with one or more pharmaceutically acceptable carriers or excipients.

In a sixth aspect of the invention, the invention relates to the dual specificity polypeptide molecule according to the invention, the nucleic acid(s) or the expression vector(s) according to the invention, the cell according to the invention, or the pharmaceutical composition according to the invention, for use in medicine.

In a seventh aspect of the invention, the invention relates to the dual specificity polypeptide molecule according to the invention, the nucleic acid or the expression vector(s) according to the invention, the cell according to the invention, or the pharmaceutical composition according to the invention, for use in the treatment of a disease or disorder as disclosed herein, in particular selected from cancer and infectious diseases.

In an eighth aspect of the invention, the invention relates to a method for the treatment of a disease or disorder comprising administering a therapeutically effective amount of the dual specificity polypeptide molecule according to the invention, the nucleic acid or the expression vector(s) according to the invention, the cell according to the invention, or the pharmaceutical composition according to the invention.

In a ninth aspect of the invention, the invention relates to a method of eliciting an immune response in a patient or subject comprising administering a therapeutically effective amount of the dual specificity polypeptide molecule according to the invention or the pharmaceutical composition according to the invention.

In a tenth aspect, the invention relates to a method of killing target cells in a patient or subject comprising administering to the patient an effective amount of the dual specificity polypeptide molecule according to the present invention.

As mentioned above, the invention provides new and improved dual specificity polypeptide molecules. The molecules generally comprise a first polypeptide chain and a second polypeptide chain, wherein the chains jointly provide a variable domain of an antibody specific for an epitope of an immune effector cell surface antigen, and a variable domain of a TCR that is specific for an MHC-associated peptide epitope, e.g. cancer epitope or epitopes presented because of infection, e.g. viral infection, such as HIV. Antibody and TCR-derived variable domains are stabilized by covalent and non-covalent bonds formed between Fc-parts or portions thereof located on both polypeptide chains. The dual specificity polypeptide molecule is then capable of simultaneously binding the cellular receptor and the MHC-associated peptide epitope.

In the context of the present invention, variable domains (VD1) and (VD2) are derived from antibodies capable of recruiting human immune effector cells by specifically binding to a surface antigen of said effector cells. In one particular embodiment, said antibodies specifically bind to epitopes of the TCR-CD3 complex of human T cells, comprising the peptide chains TCRalpha, TCRbeta, CD3gamma, CD3delta, CD3epsilon, and CD3zeta.

The dual specificity polypeptide molecule according to the present invention comprise a first polypeptide and a second polypeptide chain providing a first (VD1) and a second (VD2) binding region, respectively, of a variable domain derived from an antibody capable of recruiting human immune effector cells by specifically binding to a surface antigen of said cells. This first binding region (VD1) and said second binding region (VD2) associate to form a first binding site (VD1) (VD2) that binds the epitope of the immune effector cell surface antigen. Furthermore, the first and the second polypeptide chain of the polypeptide molecule comprises a first (VR1) and a second (VR2) binding region, respectively, of a variable domain derived from a TCR being specific for an MHC-associated peptide epitope. Said first binding region (VR1) and said second binding region (VR2) associate to form a second binding site (VR1) (VR2) that binds said MHC-associated peptide epitope. In one embodiment of the dual specificity polypeptide molecule according to the invention, the order/orientation of the regions in the first polypeptide chain is selected from VD1-LINK1-VR1, and VR1-LINK1-VD1; in another embodiment, in the order/orientation of the regions in the second polypeptide chain is selected from VD2-LINK2-VR2, and VR2-LINK-VD2, that is, the arrangement of the binding sites can be re-arranged into a “left-handed” or “right-handed” molecule (see, for example, FIG. 5). Furthermore, the configuration of the alpha and beta chains of the TCR-related part can be switched.

In the context of the present invention, the dual affinity polypeptide molecule according to the invention is exemplified by a construct that binds the SLYNTVATL peptide (SEQ ID No. 7) when presented as a peptide-MHC complex. Nevertheless, the concept of the invention is clearly not restricted to this particular peptide, and includes basically any disease- or disorder related epitope that is presented in the context with the MHC molecule. This presentation can be both MHC class-I or -II related. Major histocompatibility complex class I (MHC-I) molecules are present on the surface of all nucleated cells and display a large array of peptide epitopes for surveillance by the CD8⁺ T cell repertoire. CD8⁺ T cell responses are essential for control and clearance of viral infections as well as for the elimination of transformed and tumorigenic cells. Examples for preferred peptide epitopes to be recognized can be found in the respective literature, and especially include the peptides as disclosed in tables 1 to 5 of WO 2016/170139; tables 1 to 5 of WO 2016/102272; tables 1 or 2 of WO 2016/156202; tables 1 to 4 of WO 2016/146751; table 2 of WO 2011/113819; tables 1 to 4b of WO 2016/156230; tables 1 to 4b of WO 2016/177784; tables 1 to 4 of WO 2016/202963; tables 1 and 2 of WO 2016/207164; tables 1 to 4 of WO 2017/001491; tables 1 to 4 of WO 2017/005733; tables 1 to 8 of WO 2017/021527; tables 1 to 3 of WO 2017/036936; tables 1 to 4 of PCT/EP2016/073416 for cancer treatment(s), U.S. Publication 2016-0187351, U.S. Publication 2017-0165335, U.S. Publication 2017-0035807, U.S. Publication 2016-0280759, U.S. Publication 2016-0287687, U.S. Publication 2016-0346371, U.S. Publication 2016-0368965, U.S. Publication 2017-0022251, U.S. Publication 2017-0002055, U.S. Publication 2017-0029486, U.S. Publication 2017-0037089, U.S. Publication 2017-0136108, U.S. Publication 2017-0101473, U.S. Publication 2017-0096461, U.S. Publication 2017-0165337, U.S. Publication 2017-0189505, U.S. Publication 2017-0173132, U.S. Publication 2017-0296640, U.S. Publication 2017-0253633, and U.S. Publication 2017-0260249, the contents of each of these applications are herein incorporated by reference in their entireties. In another aspect, the dual affinity polypeptide molecule according to the invention recognizes a peptide consisting of any of those peptides described in the aforementioned patent applications.

In an aspect, the dual affinity polypeptide molecule according to the invention binds or is capable of specifically being recognized/binding to one or more peptides with an overall length of from 8 to 100 amino acids, from 8 and 30 amino acids, from 8 to 16 amino acids, preferably from 8 and 14 amino acids, namely 8, 9, 10, 11, 12, 13, 14 amino acids, in case of the elongated class II binding peptides the length can also be 15, 16, 17, 18, 19, 20, 21 or 22 amino acids. In yet another aspect, the dual affinity polypeptide molecule according to the invention binds or is capable of specifically recognizing/binding to one more peptides with an overall length of from 8 to 12 amino acids, from 8 to 10 amino acids, from 9 to 15 amino acids, from 9 to 14 amino acids, from 9 to 13 amino acids, from 9 to 12 amino acids, from 9 to 11 amino acids; from 10 to 15 amino acids, from 10 to 14 amino acids, from 10 to 13 amino acids, or from 10 to 12 amino acids.

Other suitable epitopes can be identified from databases, such as, for example, the Immune Epitope Database (available at iedb.org).

The term “human immune effector cell(s)” refers to a cell within the natural repertoire of cells in the human immune system which, when activated, is able to bring about a change in the viability of a target cell. The term “viability of a target cell” may refer within the scope of the invention to the target cell's ability to survive, proliferate and/or interact with other cells. Such interaction may be either direct, for example when the target cell contacts another cell, or indirect, for example when the target cell secretes substances which have an influence on the functioning of another distant cell. The target cell may be either native or foreign to humans. In the event that the cell is native to humans, the target cell is advantageously a cell which has undergone transformation to become a malignant cell. The native cell may additionally be a pathologically modified native cell, for example a native cell infected with an organism such as a virus, a plasmodium or a bacterium. In the event that the cell is foreign to humans, the target cell is advantageously an invading pathogen, for example an invading bacterium or plasmodium.

Preferred is the dual specificity polypeptide molecule according to the invention, wherein said first and second polypeptide chains further comprise at least one hinge domain and/or an Fc domain or portion thereof. In antibodies, the “hinge” or “hinge region” or “hinge domain” refers to the flexible portion of a heavy chain located between the CH1 domain and the CH2 domain. It is approximately 25 amino acids long, and is divided into an “upper hinge,” a “middle hinge” or “core hinge,” and a “lower hinge.” A “hinge subdomain” refers to the upper hinge, middle (or core) hinge or the lower hinge. The amino acids sequences of the hinges of an IgG1, IgG2, IgG3 and IgG4 molecule are (EU numbering indicated):

IgG1:
(SEQ ID No. 1)
E₂₁₆PKSCDKTHTCPPCPAPELLG
IgG2:
(SEQ ID No. 2)
E₂₁₆RKCCVECPPCPAPPVAGP
IgG3:
(SEQ ID No. 3)
ELKTPLGDTTHTCPRCPEPKSCDTPPPCPRCPE₂₁₆PKSCDTPPPCPRCP
APELLG
IgG4:
(SEQ ID No. 4)
E₂₁₆SKYGPPCPSCPAPEFLG

The core hinge region usually contains at least one cysteine-bridge connecting the two heavy chains. Furthermore, mutations can be made in the lower hinge region to ameliorate unwanted antibody-dependent cell-mediated cytotoxicity (ADCC).

Preferred is a dual specificity polypeptide molecule according to the present invention, comprising at least one IgG fragment crystallizable (Fc) domain, i.e. a fragment crystallizable region (Fc region), the tail region of an antibody that interacts with Fc receptors and some proteins of the complement system. Fc regions contain two or three heavy chain constant domains (CH domains 2, 3, and 4) in each polypeptide chain. The Fc regions of IgGs also bear a highly conserved N-glycosylation site. Glycosylation of the Fc fragment is essential for Fc receptor-mediated activity. The small size of bispecific antibody formats such as BiTEs® and DARTs (˜50 kD) can lead to fast clearance and a short half-life. Therefore, for improved pharmacokinetic properties, the scTv-cellular receptor (e.g. CD3) dual specificity polypeptide molecule can be fused to a (human IgG1) Fc domain, thereby increasing the molecular mass. Several mutations located at the interface between the CH2 and CH3 domains, such as T250Q/M428L and M252Y/S254T/T256E+H433K/N434F, have been shown to increase the binding affinity to neonatal Fc receptor (FcRn) and the half-life of IgG1 in vivo. By this the serum half-life of an Fc-containing molecule could be further extended.

In the dual specificity polypeptide molecules of the invention, said Fc domain can comprises a CH2 domain comprising at least one effector function silencing mutation. Preferably, these mutations are introduced into the ELLGGP (SEQ ID No. 50) sequence of human IgG1 (residues 233-238) or corresponding residues of other isotypes) known to be relevant for effector functions. In principle, one or more mutations corresponding to residues derived from IgG2 and/or IgG4 are introduced into IgG1 Fc. Preferred are: E233P, L234V, L235A and no residue or G in position 236. Another mutation is P331S. EP1075496 discloses a recombinant antibody comprising a chimeric domain which is derived from two or more human immunoglobulin heavy chain CH2 domains, which human immunoglobulins are selected from IgG1, IgG2 and IgG4, and wherein the chimeric domain is a human immunoglobulin heavy chain CH2 domain which has the following blocks of amino acids at the stated positions: 233P, 234V, 235A and no residue or G in position 236 and 327G, 330S and 331S in accordance with the EU numbering system, and is at least 98% identical to a CH2 sequence (residues 231-340) from human IgG1, IgG2 or IgG4 having said modified amino acids.

Examples of preferred CH2 partial sequences to be used can be (fully or partially) as follows:

(SEQ ID No. 5)
231-
APPVA-
GPSVFLFPPKPKDTLMISRTPEVTCVVVDVSHEDPEVKFNWYVDGVEVHN
AKTKPREEQYQSTYRVVSVLTVLHQDWLNGKEYKCKVSNKALPASIEK-
334;
and
(SEQ ID No. 6)
231-
APPVA-
GPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPASIEK-
334,

with the changes underlined, that in position 297 carry an N (glycosylated variant) or a residue selected from the group of A, G and Q (deglycosylated variant).

In the dual specificity polypeptide molecules of the invention, said Fc domain can comprise a CH3 domain comprising at least one mutation facilitating the formation of heterodimers. To maximize yield of the desired heterodimeric dual specificity-Fc protein and to simplify purification, “knobs-into-holes” mutations can be engineered into the Fc domain. With this design, Fc domains are driven to form heterodimers instead of their normal homodimers by addition of protruding bulky hydrophobic residues (“knobs”) to one chain and creation of complementary hydrophobic pockets (“holes”) on the other. A ‘knob’ variant can be obtained by replacement of a small amino acid with a larger one to insert into a ‘hole’ in the opposite domain created by replacement of a large residue with a smaller one (Ridgway, J. B. B.; Presta, L. G.; Carter, P. “Knobs-into-holes” engineering of antibody CH3 domains for heavy chain heterodimerization. Protein Eng. 1996, 9, 617-621; WO 2002/002781).

Preferred is a dual specificity polypeptide molecule according to the invention, wherein said knob-into-hole mutation is selected from T366W as knob, and T366′S, L368′A, and Y407′V as hole in the CH3 domain (see, e.g. WO 98/50431). This set of mutations can be further extended by inclusion of the mutations K409A and F405′K as described by Wei et al. (Structural basis of a novel heterodimeric Fc for bispecific antibody production, Oncotarget. 2017). Another knob can be T366Y and the hole is Y407′T.

The dual specificity polypeptide molecules of the invention can furthermore comprise artificially introduced cysteine bridges between at least one cysteine residue on the first polypeptide chain and at least one cysteine residue on the second polypeptide chain in order to improve the stability of the molecules, optimally without interfering with the binding characteristics of the bivalent molecule, and/or for improved heterodimerization. For added stability, a disulfide bond can be introduced through the addition of a single cysteine in the CH3 domain of both the knob and hole chains. Preferred is the dual specificity polypeptide molecule according to the invention, wherein the Fc domain comprises a CH3 domain comprising at least one additional cysteine residue, for example S354C and/or Y349C.

Preferred is a dual specificity polypeptide molecule according to the invention wherein said CD molecule is selected from the group of immune response-related CD molecules, CD3, such as the CD3γ, CD3δ, and CD3ε chains, CD4, CD7, CD8, CD10, CD11b, CD11c, CD14, CD16, CD18, CD22, CD25, CD28, CD32a, CD32b, CD33, CD41, CD41b, CD42a, CD42b, CD44, CD45RA, CD49, CD55, CD56, CD61, CD64, CD68, CD94, CD90, CD117, CD123, CD125, CD134, CD137, CD152, CD163, CD193, CD203c, CD235a, CD278, CD279, CD287, Nkp46, NKG2D, GITR, FcεRI, TCRalpha/beta, TCRgamma/delta and HLA-DR. Depending on the combination of the two antigen binding entities of the dual specificity polypeptide molecule according to the invention, specific advantages regarding the function of the molecule, in particular an enhanced activity can be achieved.

Preferred is the exemplary dual specificity polypeptide molecule according to the invention, wherein the regions in the first polypeptide chain SEQ ID No. 28 for VR1, SEQ ID No. 29 for VD1, SEQ ID No. 30 for LINK1; and the regions in the second polypeptide chain comprise SEQ ID No. 31 for VR2, SEQ ID No. 32 for VD2, and SEQ ID No. 30 for LINK2.

Further preferred is the exemplary dual specificity polypeptide molecule according to the invention, wherein the FC region in the first polypeptide chain comprises SEQ ID No. 26 (Fc1), and the FC region in the second polypeptide chain comprises SEQ ID No. 27 (Fc2).

Further preferred is the exemplary dual specificity polypeptide molecule according to the invention comprising a first polypeptide chain comprising SEQ ID No. 16 (1. chain of full molecule) and a second polypeptide chain comprising SEQ ID No. 17 (2. chain of full molecule).

Even further preferred is the exemplary dual specificity polypeptide molecule according to the invention, wherein said first binding site (VD1) (VD2) that binds the epitope of the surface antigen of human immune cells (e.g. CD3) is humanized; and/or said second binding site (VR1) (VR2) that binds said MHC-associated peptide epitope is affinity maturated.

Humanized antibodies are antibodies (or parts thereof) from non-human species whose protein sequences have been modified to increase their similarity to antibody variants produced naturally in humans. The process of “humanization” is usually applied to monoclonal antibodies developed for administration to humans (for example, antibodies developed as anti-cancer drugs). Suitable methods for humanization are known from the literature, and, for example, reviewed in Olimpieri, Pier Paolo, Paolo Marcatili, and Anna Tramontano. “Tabhu: Tools for Antibody Humanization.” Bioinformatics 31.3 (2015): 434-435. PMC; Safdari Y, Farajnia S, Asgharzadeh M, Khalili M. Antibody humanization methods—a review and update. Biotechnol Genet Eng Rev. 2013; 29:175-86; or Ahmadzadeh V, Farajnia S, Feizi M A, Nejad R A. Antibody humanization methods for development of therapeutic applications. Monoclon Antib Immunodiagn Immunother. 2014 April; 33 (2): 67-73.

In general, in vitro affinity maturation of TCRs and antibodies can be done according to methods described in the literature, in particular using yeast or phage surface display (based on, for example, Holler P D, et al. In vitro evolution of a T cell receptor with high affinity for peptide/MHC. Proc Natl Acad Sci USA. 2000 May 9; 97 (10): 5387-92; Boder E T et al., Directed evolution of antibody fragments with monovalent femtomolar antigen-binding affinity. Proc Natl Acad Sci USA. 2000 Sep. 26; 97 (20): 10701-5; and, as a recent example, Zhao Q, et al. Affinity maturation of T-cell receptor-like antibodies for Wilms tumor 1 peptide greatly enhances therapeutic potential. Leukemia. 2015; 29 (11): 2238-2247).

The binding sites (VD1) (VD2) and (VR1) (VR2) of the present description preferably specifically bind to a surface antigen of human immune cells and a peptide-HLA molecule complex, respectively. As used herein in connection with binding sites of the present description, “specific binding” and grammatical variants thereof are used to mean a site having a binding affinity (KD) for a peptide-HLA molecule complex and/or an antibody epitope of 100 μM or less. The binding sites (VD1) (VD2) and (VR1) (VR2) of the present description bind to a CD antibody epitope or a peptide-HLA molecule complex, respectively, with a binding affinity (KD) of about 100 μM or less, about 50 μM or less, about 25 μM or less, or about 10 μM or less. More preferred are high affinity binding sites having binding affinities of about 1 μM or less, about 100 nM or less, about 50 nM or less, about 25 nM or less, about 10 nM or less, about 5 nM or less, about 2 nM or less, about 1 nM or less, about 500 pM or less, about 200 pM or less, about 100 pM or less Non-limiting examples of preferred binding affinity ranges for binding sites of the present invention include about 10 pM to about 100 pM, 100 pM to about 1 nM, 1 nM to about 10 nM; about 10 nM to about 20 nM; about 20 nM to about 30 nM; about 30 nM to about 40 nM; about 40 nM to about 50 nM; about 50 nM to about 60 nM; about 60 nM to about 70 nM; about 70 nM to about 80 nM; about 80 nM to about 90 nM; and about 90 nM to about 100 nM, e.g. measured by Bio-Layer Interferometry as described in Example 6.

In an aspect, the disclosure provides for a polypeptide having at least 50%, at least 60%, at least 70%, at least 80%, at least 90%, at least 95%, at least 96%, at least 97%, at least 98%, or at least 99% sequence identity to an amino acid sequence described herein, for example, amino acid sequences 1 to 58. In another aspect, the disclosure provides for a first or second polypeptide having at least 50%, at least 60%, at least 70%, at least 80%, at least 90%, at least 95%, at least 96%, at least 97%, at least 98%, or at least 99% sequence identity to an amino acid sequence described herein. In yet another aspect, the disclosure provides for a duel specific polypeptide molecule having a sequence identity of at least 50%, at least 60%, at least 70%, at least 80%, at least 90%, at least 95%, at least 96%, at least 97%, at least 98%, or at least 99% sequence identity to one or more amino acid sequences described herein. The disclosure further provides for aspects wherein the percent identity of at least 50%, at least 60%, at least 70%, at least 80%, at least 90%, at least 95%, at least 96%, at least 97%, at least 98%, or at least 99% applies to any of the sequences of the structural regions described in FIG. 1, for example, VD1, VR1, Link1, VR2, VD2, Link2, or hinge region, and as described or being part of the sequences as disclosed herein.

In an aspect, polypeptides or duel specific polypeptide molecules described herein may be altered in various ways including amino acid substitutions, deletions, truncations, and insertions of one or more amino acids. In another aspect, polypeptides or duel specific polypeptide molecules described herein may include 1, 2, 3, 4, 5, 6, 7, 8, 9, 10, 15, 20, 25, 30, 35, 40, 45, or 50 or more amino acid substitutions, deletions or insertions. In yet another aspect, polypeptides or duel specific polypeptide molecules described herein may include 1 to 5, 1 to 10, 1 to 20, 2 to 5, 2 to 10, 5 to 20, 5 to 50, or 10 to 100 amino acid substitutions, deletions or insertions. In an aspect, 1, 2, 3, 4, 5, 6, 7, 8, 9, 10, 15, 20, 25, 30, 35, 40, 45, or 50, or more amino acid substitutions, deletions or insertions applies to any of the structural regions described in FIG. 1, for example, VD1, VR1, Link1, VR2, VD2, Link2, or hinge regions. The disclosure further provides for aspects wherein 1 to 5, 1 to 10, 1 to 20, 2 to 5, 2 to 10, 5 to 20, 5 to 50, or 10 to 100 amino acid substitutions, deletions or insertions applies to the sequences of any of the structural regions described in FIG. 1, for example, VD1, VR1, Link1, VR2, VD2, Link2, or hinge region, and as described or being part of the sequences as disclosed herein.

In an aspect, 1, 2, 3, 4, 5, 6, 7, 8, 9, 10, 15, 20, 25, 30, 35, 40, 45, or 50 or more amino acids may be added to the N-terminus or C-Terminus of a polypeptide or duel specific polypeptide molecule described herein, for example, amino acid sequences 1 to 58.

In an aspect, VR1 may have at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the amino acid sequence of SEQ ID NO: 28.

In an aspect, VD1 may have at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the amino acid sequence of SEQ ID NO: 29.

In an aspect, LINK1 or LINK2 may have at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the amino acid sequence of SEQ ID NO: 30.

In an aspect, VR2 may have at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the amino acid sequence of SEQ ID NO: 31.

In an aspect, VD2 may have at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the amino acid sequence of SEQ ID NO: 32.

In an aspect, hinge may have at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the amino acid sequence of SEQ ID NO: 1, SEQ ID NO: 2, SEQ ID NO: 3, or SEQ ID NO: 4. In an aspect, CH2 domain may have at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the amino acid sequence of SEQ ID NO: 5 or SEQ ID NO: 6.

In an aspect, Fc region may have at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the amino acid sequence of SEQ ID NO: 26 or SEQ ID NO: 27.

In an aspect, the disclosure provides for a polypeptide having at least 50%, at least 60%, at least 70%, at least 80%, at least 90%, at least 95%, at least 96%, at least 97%, at least 98%, or at least 99% sequence identity to the amino acid sequence of SEQ ID NO: 43, 44, 45, or 46.

In an aspect, the polypeptides or duel specific polypeptide molecules as disclosed herein can be modified by the substitution of one or more residues at different, possibly selective, sites within the polypeptide chain. Such substitutions may be of a conservative nature, for example, where one amino acid is replaced by an amino acid of similar structure and characteristics, such as where a hydrophobic amino acid is replaced by another hydrophobic amino acid. Even more conservative would be replacement of amino acids of the same or similar size and chemical nature, such as where leucine is replaced by isoleucine. In studies of sequence variations in families of naturally occurring homologous proteins, certain amino acid substitutions are more often tolerated than others, and these are often show correlation with similarities in size, charge, polarity, and hydrophobicity between the original amino acid and its replacement, and such is the basis for defining “conservative substitutions.”

In another preferred embodiment of the dual specificity polypeptide molecule according to the invention, said molecule carries an active agent or a portion thereof that is coupled or conjugated thereto. Said active agent can be selected from the group consisting of a detectable label, an immunostimulatory molecule, and a therapeutic agent.

The detectable label can be selected from the group consisting of biotin, streptavidin, an enzyme or catalytically active fragment thereof, a radionuclide, a nanoparticle, a paramagnetic metal ion, or a fluorescent, phosphorescent, or chemiluminescent molecule. Detectable labels for diagnostic purposes include for instance, fluorescent labels, radiolabels, enzymes, nucleic acid probes and contrast reagents.

Therapeutic agents which may be associated with the molecules of the invention include immunomodulators, radioactive compounds, enzymes (perforin for example), chemotherapeutic agents (cis-platin for example), or a toxin. Other suitable therapeutic agents include small molecule cytotoxic agents, i.e. compounds with the ability to kill mammalian cells having a molecular weight of less than 700 Daltons. Such compounds could also contain toxic metals capable of having a cytotoxic effect. Furthermore, it is to be understood that these small molecule cytotoxic agents also include pro-drugs, i.e. compounds that decay or are converted under physiological conditions to release cytotoxic agents. Examples of such agents include cis-platin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetreate glucuronate, auristatin E vincristine and doxorubicin; peptide cytotoxins, i.e. proteins or fragments thereof with the ability to kill mammalian cells. For example, ricin, diphtheria toxin, pseudomonas bacterial exotoxin A, DNase and RNase; radio-nuclides, i.e. unstable isotopes of elements which decay with the concurrent emission of one or more of α or β particles, or γ rays. For example, iodine-131, rhenium-186, indium-111, yttrium-90, bismuth-210 and -213, actinium-225 and astatine-213; chelating agents may be used to facilitate the association of these radio-nuclides to the molecules, or multimers thereof; immuno-stimulants, i.e. immune effector molecules which stimulate immune response. For example, cytokines such as IL-2 and IFN-v, chemokines such as IL-8, platelet factor 4, melanoma growth stimulatory protein, complement activators; or xenogeneic protein domains, allogeneic protein domains, viral/bacterial protein domains, viral/bacterial peptides.

Another aspect of the present invention then relates to a nucleic acid molecule encoding for a first polypeptide chain and/or a second polypeptide chain as disclosed herein, or an expression vector comprising such a nucleic acid. The nucleic acid molecule can be a DNA, cDNA, PNA, RNA, and combinations thereof. The nucleotide sequence coding for a particular peptide, oligopeptide, or polypeptide may be naturally occurring or they may be synthetically constructed. Generally, DNA segments encoding the peptides, polypeptides, and proteins of this invention are assembled from cDNA fragments and short oligonucleotide linkers, or from a series of oligonucleotides, to provide a synthetic gene that is capable of being expressed in a recombinant transcriptional unit comprising regulatory elements derived from a microbial or viral operon. The term “expression product” means the polypeptide or protein that is the natural translation product of the gene and any nucleic acid sequence coding equivalents resulting from genetic code degeneracy and thus coding for the same amino acid(s). The term “fragment”, when referring to a coding sequence, means a portion of DNA comprising less than the complete coding region, whose expression product retains essentially the same biological function or activity as the expression product of the complete coding region. Depending on the intended use, the nucleic acid can be codon-optimized for expression in a suitable (e.g. microbial) host cell. Redundancy in the genetic code allows some amino acids to be encoded by more than one codon, but certain codons are less “optimal” than others because of the relative availability of matching tRNAs as well as other factors (Gustafsson et al., 2004).

The nucleic acid may be, for example, DNA, cDNA, PNA, RNA or combinations thereof, either single- and/or double-stranded, or native or stabilized forms of polynucleotides, such as, for example, polynucleotides with a phosphorothioate backbone and may or may not contain introns so long as it codes for the polypeptide chains.

The nucleic acid (e.g. DNA) may then be comprised and/or expressed in a suitable host to produce a polypeptide comprising the polypeptide chain of the invention. Thus, the nucleic acid (e.g. DNA) encoding the polypeptide chain of the invention may be used in accordance with known techniques, appropriately modified in view of the teachings contained herein, to construct an expression vector, which is then used to transform an appropriate host cell for the expression and production of the polypeptide of the invention, as is known in the art. The nucleic acid (e.g. DNA, or in the case of retroviral vectors, RNA) encoding the polypeptide chain(s) constituting the compound of the invention may be joined to a wide variety of other nucleic acid (e.g. DNA) sequences for introduction into an appropriate host. The companion nucleic acid will depend upon the nature of the host, the manner of the introduction of the DNA into the host, and whether episomal maintenance or integration is desired. Generally, the nucleic acid is inserted into an expression vector, such as a plasmid, in proper orientation and correct reading frame for expression. If necessary, the nucleic acid may be linked to the appropriate transcriptional and translational regulatory control nucleotide sequences recognized by the desired host, although such controls are generally available in the expression vector. The vector is then introduced into the host using standard techniques. Generally, not all of the hosts will be transformed by the vector. Therefore, it will be necessary to select for transformed host cells. One selection technique involves incorporating into the expression vector a nucleic acid sequence, with any necessary control elements, that codes for a selectable trait in the transformed cell, such as antibiotic resistance. Alternatively, the gene for such selectable trait can be on another vector, which is used to co-transform the desired host cell. Host cells that have been transformed by the recombinant nucleic acid of the invention are then cultured for a sufficient time and under appropriate conditions known to those skilled in the art in view of the teachings disclosed herein to permit the expression of the polypeptide, which can then be recovered.

Many expression systems are known, including bacteria (for example E. coli and Bacillus subtilis), yeasts (for example Saccharomyces cerevisiae), filamentous fungi (for example Aspergillus spec), plant cells, animal cells and insect cells. Preferably, the system can be mammalian cells such as CHO cells available from the ATCC Cell Biology Collection.

In one embodiment, the description provides a method of producing a molecule as described herein, the method comprising culturing a host cell capable of expressing the polypeptide chain(s) under conditions suitable to promote expression of said chain(s).

In one aspect, to obtain cells expressing molecules of the present description, nucleic acids encoding polypeptide chains comprising TCR-alpha and/or TCR-beta binding domains are cloned into expression vectors, such as gamma retrovirus or lentivirus. In another aspect, to obtain cells expressing molecules of the present description, RNAs are synthesized by techniques known in the art, e.g., in vitro transcription systems. The in vitro-synthesized RNAs are then introduced into suitable cells by electroporation to express polypeptide chains.

To increase the expression, nucleic acids encoding chains of the present description may be operably linked to strong promoters, such as retroviral long terminal repeats (LTRs), cytomegalovirus (CMV), murine stem cell virus (MSCV) U3, phosphoglycerate kinase (PGK), β-actin, ubiquitin, and a simian virus 40 (SV40)/CD43 composite promoter, elongation factor (EF)-1a and the spleen focus-forming virus (SFFV) promoter. In a preferred embodiment, the promoter is heterologous to the nucleic acid being expressed. In addition to strong promoters, expression cassettes of the present description may contain additional elements that can enhance transgene expression, including a central polypurine tract (cPPT), which promotes the nuclear translocation of lentiviral constructs (Follenzi et al., 2000), and the woodchuck hepatitis virus posttranscriptional regulatory element (wPRE), which increases the level of transgene expression by increasing RNA stability (Zufferey et al., 1999).

The alpha and beta binding domain chains of a molecule of the present invention may be encoded by nucleic acids located in separate vectors, or may be encoded by polynucleotides located in the same vector.

In an embodiment, a host cell is engineered to express a molecule of the present description. Host cells of the present description can be allogeneic or autologous with respect to a patient to be treated.

Yet another aspect of the invention relates to a pharmaceutical composition comprising the dual specificity polypeptide molecule according to the present invention, the nucleic acid(s) or the expression vector(s) according to the present invention, or the cell according to the present invention, together with one or more pharmaceutically acceptable carriers or excipients. The compositions of the invention include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of the prophylactic and/or therapeutic dual specificity polypeptide molecule (agent) disclosed herein or a combination of the agent and a pharmaceutically acceptable carrier. Preferably, compositions of the invention comprise a prophylactically or therapeutically effective amount of one or more molecules of the invention and a pharmaceutically acceptable carrier.

The pharmaceutical compositions preferably comprise the molecules either in the free form or as a salt. Preferably, the salts are pharmaceutical acceptable salts of the molecules, such as, for example, the chloride or acetate (trifluoroacetate) salts. It has to be noted that the salts of the molecules according to the present invention differ substantially from the molecules in their state(s) in vivo, as the molecules are not salts in vivo.

An embodiment of the present invention thus relates to a non-naturally occurring molecule according to the invention that has been synthetically produced (e.g. synthesized) as a pharmaceutically acceptable salt. Methods to synthetically produce peptides and/or polypeptides are well known in the art. The salts of the molecules according to the present invention differ substantially from the molecules in their state(s) in vivo, as the molecules as generated in vivo are no salts. Preferably, the salts are pharmaceutically acceptable salts of the molecules. These salts according to the invention include alkaline and earth alkaline salts such as salts of the Hofmeister series comprising as anions PO₄³⁻, SO₄²⁻, CH₃COO⁻, Cl⁻, Br⁻, NO₃⁻, ClO₄⁻, I⁻, SCN⁻ and as cations NH₄⁺, Rb⁺, K⁺, Na⁺, Cs⁺, Li⁺, Zn²⁺, Mg²⁺, Ca²⁺, Mn²⁺, Cu²⁺ and Ba²⁺. Particularly salts are selected from (NH₄)₃PO₄, (NH₄)₂HPO₄, (NH₄)H₂PO₄, (NH₄)₂SO₄, NH₄CH₃COO, NH₄Cl, NH₄Br, NH₄NO₃, NH₄ClO₄, NH₄I, NH₄SCN, Rb₃PO₄, Rb₂HPO₄, Rb₂HPO₄, Rb₂SO₄, Rb₄CH₃COO, Rb₄Cl, Rb₄Br, Rb₄NO₃, Rb₄ClO₄, Rb₄I, Rb₄SCN, K₃PO₄, K₂HPO₄, KH₂PO₄, K₂SO₄, KCH₃COO, KCl, KBr, KNO₃, KClO₄, KI, KSCN, Na₃PO₄, Na₂HPO₄, NaH₂PO₄, Na₂SO₄, NACH₃COO, NaCl, NaBr, NaNO₃, NaClO₄, NaI, NaSCN, ZnCl₂Cs₃PO₄, Cs₂HPO₄, CsH₂PO₄, Cs₂SO₄, CsCH₃COO, CsCl, CsBr, CsNO₃, CsClO₄, CsI, CsSCN, Li₃PO₄, Li₂HPO₄, LiH₂PO₄, Li₂SO₄, LiCH₃COO, LiCl, LiBr, LiNO₃, LiClO₄, LiI, LiSCN, Cu₂SO₄, Mg₃(PO₄)₂, Mg₂HPO₄, Mg(H₂PO₄)₂, Mg₂SO₄, Mg(CH₃COO)₂, MgCl₂, MgBr₂, Mg(NO₃)₂, Mg(ClO₄)₂, MgI₂, Mg(SCN)₂, MnCl₂, Ca₃(PO₄)₂, Ca₂HPO₄, Ca(H₂PO₄)₂, CaSO₄, Ca(CH₃COO)₂, CaCl₂), CaBr₂, Ca(NO₃)₂, Ca(ClO₄)₂, CaI₂, Ca(SCN)₂, Ba₃(PO₄)₂, Ba₂HPO₄, Ba(H₂PO₄)₂, BaSO₄, Ba(CH₃COO)₂, BaCl₂, BaBr₂, Ba(NO₃)₂, Ba(ClO₄)₂, BaI₂, and Ba(SCN)₂. Particularly preferred are NH acetate, MgCl₂, KH₂PO₄, Na₂SO₄, KCl, NaCl, and CaCl₂), such as, for example, the chloride or acetate (trifluoroacetate) salts.

In an aspect, a polypeptide described herein is in the form of a pharmaceutically acceptable salt. In another aspect, a polypeptide in the form of a pharmaceutical salt is in crystalline form.

In an aspect, a pharmaceutically acceptable salt described herein refers to salts which possess toxicity profiles within a range that is acceptable for pharmaceutical applications.

As used herein, “a pharmaceutically acceptable salt” refers to a derivative of the disclosed peptides wherein the peptide is modified by making acid or base salts of the agent. For example, acid salts are prepared from the free base (typically wherein the neutral form of the drug has a neutral —NH₂ group) involving reaction with a suitable acid. Suitable acids for preparing acid salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methane sulfonic acid, ethane sulfonic acid, p-toluenesulfonic acid, salicylic acid, and the like, as well as inorganic acids, e.g., hydrochloric acid, hydrobromic acid, sulfuric acid, nitric acid phosphoric acid and the like. Conversely, preparation of basic salts of acid moieties which may be present on a peptide are prepared using a pharmaceutically acceptable base such as sodium hydroxide, potassium hydroxide, ammonium hydroxide, calcium hydroxide, trimethylamine or the like.

In an aspect, pharmaceutically acceptable salts may increase the solubility and/or stability of peptides of described herein. In another aspect, pharmaceutical salts described herein may be prepared by conventional means from the corresponding carrier peptide or complex by reacting, for example, the appropriate acid or base with peptides or complexes as described herein. In another aspect, the pharmaceutically acceptable salts are in crystalline form or semi-crystalline form. In yet another aspect, pharmaceutically acceptable salts may include, for example, those described in Handbook of Pharmaceutical Salts: Properties, Selection, and Use by P. H. Stahl and C. G. Wermuth (Wiley-VCH 2002) and L. D. Bighley, S. M. Berge, D. C. Monkhouse, in “Encyclopedia of Pharmaceutical Technology”. Eds. J. Swarbrick and J. C. Boylan, Vol. 13, Marcel Dekker, Inc., New York, Basel, Hong Kong 1995, pp. 453-499, each of these references is herein incorporated by reference in its entirety.

The invention also encompasses pharmaceutical compositions comprising a dual specificity polypeptide molecule of the invention and a therapeutic antibody (e.g., tumor specific monoclonal antibody) that is specific for a particular cancer antigen, and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, sodium phosphate, sodium acetate, L-Histidine, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Generally, the ingredients of compositions of the invention are supplied either separately or mixed together in unit dosage form, for example, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Another aspect of the present invention then relates to the dual specificity polypeptide molecule according to the invention, the nucleic acid or the expression vector according to the invention, the cell according to the invention, or the pharmaceutical composition according to the invention, for use in medicine. In general, the use of the dual specificity polypeptide molecule depends on the medical context of the peptide-antigen(s) that is/are recognized by said molecule, as is also described further below.

Preferred is the dual specificity polypeptide molecule according to the invention, the nucleic acid or the expression vector according to the invention, or the cell according to the invention, or the pharmaceutical composition according to the invention, for use in the treatment or prevention of a disease or disorder selected from immunological disorders, infectious disease, intoxication and cancers, including treatment or prevention of a variety of cancers or other abnormal proliferative diseases, including (but not limited to) the following: carcinoma, including that of the bladder, breast, colon, kidney, liver, lung, ovary, pancreas, stomach, prostate, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burketts' lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, teratocarcinoma, neuroblastoma and glioma; tumors of the central and peripheral nervous system, including astrocytoma, neuroblastoma, glioma, schwannomas, and osteosarcoma; and other tumors, including melanoma, xeroderma pigmentosum, keratoactanthoma, seminoma, thyroid follicular cancer, and teratocarcinoma. Additional cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented by the methods and compositions of the invention in the ovary, bladder, breast, colon, lung, skin, pancreas, or uterus. In other specific embodiments, sarcoma, melanoma, or leukemia is treated or prevented by the methods and compositions of the invention.

The invention further relates to methods of eliciting an immune response in a patient or subject comprising administering a therapeutically effective amount of the dual specificity polypeptide molecule according to the invention or the pharmaceutical composition according to the invention. In an aspect, a population of the dual specificity polypeptide molecule according to the invention or the pharmaceutical composition according to the invention is administered to a patient or subject in need thereof.

The invention further relates to a method of killing target cells in a patient or subject comprising administering to the patient an effective amount of the dual specificity polypeptide molecule according to the present invention.

The invention also provides methods for preventing, treating, or managing one or more symptoms associated with an inflammatory disorder in a subject further comprising, administering to said subject a therapeutically or prophylactically effective amount of one or more anti-inflammatory agents according to the invention. The invention also provides methods for preventing, treating, or managing one or more symptoms associated with an autoimmune disease further comprising, administering to said subject a therapeutically or prophylactically effective amount of one or more immunomodulatory agents according to the invention. Infectious diseases that can be treated or prevented by the molecules of the invention are caused by infectious agents including but not limited to viruses, bacteria, fungi, protozoae, and viruses. Viral diseases that can be treated or prevented using the molecules of the invention in conjunction with the methods of the present invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, hantavirus, coxsackie virus, mumps virus, measles virus, rubella virus, polio virus, small pox, Epstein Barr virus, human immunodeficiency virus type I (HIV-I), human immunodeficiency virus type II (HIV-II), and agents of viral diseases such as viral meningitis, encephalitis, dengue or small pox.

Bacterial diseases that can be treated or prevented using the molecules of the invention in conjunction with the methods of the present invention, that are caused by bacteria include, but are not limited to, mycobacteria rickettsia, mycoplasma, Neisseria, S. pneumonia, Borrelia burgdorferi (Lyme disease), Bacillus antracis (anthrax), tetanus, streptococcus, staphylococcus, mycobacterium, tetanus, pertussis, cholera, plague, diphtheria, chlamydia, S. aureus and legionella.

Protozoal diseases that can be treated or prevented using the molecules of the invention in conjunction with the methods of the present invention, that are caused by protozoa include, but are not limited to, leishmania, kokzidioa, trypanosoma or malaria. Parasitic diseases that can be treated or prevented using the molecules of the invention in conjunction with the methods of the present invention, that are caused by parasites include, but are not limited to, chlamydia and rickettsia.

Examples of infectious agents and diseases include but are not limited to bacteria (e.g., Escherichia coli, Klebsiella pneumoniae, Staphylococcus aureus, Enterococcus faecalis, Candida albicans, Proteus vulgaris, Staphylococcus viridans, and Pseudomonas aeruginosa), a pathogen (e.g., B-lymphotropic papovavirus (LPV); Bordetella pertussis; Borna Disease virus (BDV); Bovine coronavirus; Choriomeningitis virus; Dengue virus; a virus, E. coli; Ebola; Echovirus 1; Echovirus-11 (EV); Endotoxin (LPS); Enteric bacteria; Enteric Orphan virus; Enteroviruses; Feline leukemia virus; Foot and mouth disease virus; Gibbon ape leukemia virus (GALV); Gram-negative bacteria; Helicobacter pylorii; Hepatitis B virus (HBV); Herpes Simplex Virus; HIV-I; Human cytomegalovirus; Human coronovirus; Influenza A, B and C; Legionella; Leishmania mexicana; Listeria monocytogenes; Measles virus; Meningococcus; Morbilliviruses; Mouse hepatitis virus; Murine leukemia virus; Murine gamma herpes virus; Murine retrovirus; Murine coronavirus mouse hepatitis virus; Mycobacterium avium-M; Neisseria gonorrhoeae; Newcastle disease virus; Parvovirus B 19; Plasmodium falciparum; Pox Virus; Pseudomonas; Rotavirus; Salmonella typhiurium; Shigella; Streptococci; T-cell lymphotropic virus 1; Vaccinia virus).

Yet another aspect of the present invention then relates to a method for the treatment of a disease or disorder comprising administering a therapeutically effective amount of the dual specificity polypeptide molecule according to the invention, the nucleic acid or the expression vector according to the invention, the cell according to the invention, or the pharmaceutical composition according to the invention.

The dual specificity polypeptide molecule of the invention may be used in a method of preventing or treating a disease or condition which is ameliorated by administration of the dual specificity polypeptide molecule. Such treatments may be provided in a pharmaceutical composition together with one or more pharmaceutically acceptable carriers or excipients. Therapeutic dual specificity polypeptide molecules will usually be supplied as part of a sterile, pharmaceutical composition which will normally include a pharmaceutically acceptable carrier. This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms. The pharmaceutical composition may be adapted for administration by any appropriate route, such as a parenteral (including subcutaneous, intramuscular, or intravenous) route. Such compositions may be prepared by any method known in the art of pharmacy, for example by mixing the active ingredient with the carrier(s) or excipient(s) under sterile conditions.

In an aspect, peptides or other molecules described herein may be combined with an aqueous carrier. In an aspect, the aqueous carrier is selected from ion exchangers, alumina, aluminum stearate, magnesium stearate, lecithin, serum proteins, such as human serum albumin, buffer substances such as phosphates, glycine, sorbic acid, potassium sorbate, partial glyceride mixtures of saturated vegetable fatty acids, salts or electrolytes, such as protamine sulfate, disodium hydrogen phosphate, dicalcium phosphate, potassium hydrogen phosphate, sodium chloride, zinc salts, colloidal silica, magnesium trisilicate, polyvinyl pyrrolidone, polyvinylpyrrolidone-vinyl acetate, cellulose-based substances (e.g., microcrystalline cellulose, hydroxypropyl methylcellulose, hydroxypropyl methylcellulose acetate succinate, hydroxypropyl methylcellulose Phthalate), starch, lactose monohydrate, mannitol, trehalose sodium lauryl sulfate, and crosscarmellose sodium, polyethylene glycol, sodium carboxymethylcellulose, polyacrylates, polymethacrylate, waxes, polyethylene-polyoxypropylene-block polymers, polyethylene glycol and wool fat.

In an aspect, the aqueous carrier contains multiple components, such as water together with a non-water carrier component, such as those components described herein. In another aspect, the aqueous carrier is capable of imparting improved properties when combined with a peptide or other molecule described herein, for example, improved solubility, efficacy, and/or improved immunotherapy. In addition, the composition can contain excipients, such as buffers, binding agents, blasting agents, diluents, flavors, lubricants, etc. A “pharmaceutically acceptable diluent,” for example, may include solvents, bulking agents, stabilizing agents, dispersion media, coatings, antibacterial and antifungal agents, isotonic and absorption delaying agents, and the like which are physiologically compatible. Examples of pharmaceutically acceptable diluents include one or more of saline, phosphate buffered saline, dextrose, glycerol, ethanol, and the like as well as combinations thereof. In many cases it will be preferable to include one or more isotonic agents, for example, sugars such as trehalose and sucrose, polyalcohols such as mannitol, sorbitol, or sodium chloride in the composition. Pharmaceutically acceptable substances such as wetting or minor amounts of auxiliary substances such as wetting or emulsifying agents, preservatives or buffers, are also within the scope of the present invention. In addition, the composition can contain excipients, such as buffers, binding agents, blasting agents, diluents, flavors, and lubricants.

Dosages of the dual specificity polypeptide molecules of the present invention can vary between wide limits, depending upon the disease or disorder to be treated, the age and condition of the individual to be treated, etc.; for example, a suitable dose range for a dual specificity polypeptide molecule may be between 25 ng/kg and 50 μg/kg. A physician will ultimately determine appropriate dosages to be used.

Pharmaceutical compositions, vectors, nucleic acids and cells of the invention may be provided in substantially pure form, for example at least 80%, at least 85%, at least 90%, at least 91%, at least 92%, at least 93%, at least 94%, at least 95%, at least 96%, at least 97%, at least 98%, at least 99% or 100% pure.

Preferred features of each aspect of the invention are as for each of the other aspects mutatis mutandis. The prior art documents mentioned herein are incorporated to the fullest extent permitted by law. Although the present invention and its advantages have been described in detail, it should be understood that various changes, substitutions and alterations can be made herein without departing from the spirit and scope of the invention as defined in the appended claims. The present invention will be further illustrated in the following Examples which are given for illustration purposes only and are not intended to limit the invention in any way.

### BRIEF DESCRIPTION OF THE DRAWINGS

FIG. 1 shows a schematic overview over a preferred embodiment of the present invention, the human IgG1 Fc-containing dual specificity polypeptide molecule. VD1, VD2=variable domains derived from antibody; VR1, VR2=variable domains derived from TCR; Link1, Link2=connecting linkers; Cys-Cys=cysteine bridges.

FIG. 2 shows a schematic overview over 4 different constructs of IgG Fc-containing dual specificity polypeptide molecules as tested in the context of the present invention. black=TCR-derived variable domains; light gray=antibody-derived variable domains; white=constant domains derived from human IgG. Knob-hole mutations are indicated by a cylinder. Diabody molecules IA-ID are according to the invention.

FIG. 3 shows the HPLC-SEC analysis of different bispecific TCR/mAb molecules with a molecular design according to the constructs depicted in FIG. 2, which were purified by a 2-column purification process. The monomer contents of the different molecules were determined as follows. II: 93.84%; III: 96.54%; IV: 98.49%; IA_1: 95.48%; IA_3: 98.45%; ID_1: 95.75%; IC_4: 95.22%; IC_5: 92.76%; ID_4: 99.31%; ID_5: 99.44%.

FIG. 4 shows the results of the potency assay with different bispecific TCR/mAb constructs (as shown in FIG. 2) designed as IgG4-based molecules. Jurkat_NFATRE_luc2 cells were co-incubated with HIV-peptide SLYNTVATL (SEQ ID No. 7) loaded T2 cells in the presence of increasing concentrations of bispecific TCR (bssTCR) molecules. The bispecific TCR/mAb diabody molecule IA-IgG4 exhibited a higher potency than two alternative dual specificity TCR/mAb molecules.

FIG. 5 shows the results of the potency assay with different bispecific TCR/mAb constructs (as shown in FIG. 2) designed as IgG1-based molecules. Jurkat_NFATRE_luc2 cells were co-incubated with HIV-peptide SLYNTVATL (SEQ ID No. 7) loaded T2 cells in the presence of increasing concentrations of bispecific TCR (bssTCR) molecules. The bispecific TCR/mAb diabody molecules ID_1, IA_3 and IA1 exhibited markedly higher potency than three alternative dual specificity TCR/mAb molecules.

FIG. 6 shows the results of the potency assay conducted with different IgG1-based bispecific TCR/mAb constructs (as shown in FIG. 2) utilizing different variable antibody domains both targeting the TCR-CD3 complex. Construct ID_1 comprises variable domains of the UCHT1 (V9) antibody targeting CD3, whereas the constructs ID_4 and ID_5 comprise variable domains of the alpha/beta TCR-specific antibody BMA031. Jurkat_NFATRE_luc2 cells were co-incubated with HIV-peptide SLYNTVATL (SEQ ID No. 7) loaded T2 cells in the presence of increasing concentrations of bispecific TCR (bssTCR) molecules.

FIGS. 7A, 7B, 7C, and 7D show a schematic overview over the possible orientations of the VD and VR domains in the molecules of the present invention. VH: antibody-derived VH-domain, VL: antibody-derived VL-domain; Vα: TCR-derived Valpha; Vβ: TCR-derived Vbeta.

FIG. 8 shows the results of HPLC-SEC analysis of aggregates (HMWS-high molecular weight species) within different bispecific TCR/mAb molecules based on IgG1. Aggregates were analyzed after purification and after storage of the molecules at 40° C. for 1 weeks and 2 weeks, respectively.

FIG. 9 shows the results of the potency assay conducted with different bispecific TCR/mAb molecules based on IgG1. Potency was analyzed after purification and after storage of the molecules at 40° C. for 1 week and 2 weeks, respectively. Stress storage at 40° C. did not lead to significant loss of potency of the molecules but a drastic increase in unspecific (i.e. target-independent) activation of Jurkat T cells was detected for the molecules III and IV.

FIG. 10 shows the results of a LDH-release assay with different bispecific TCR/mAb constructs (as shown in FIG. 2) designed as IgG1-based molecules. PBMC isolated from a healthy donor were co-incubated with HIV-peptide SLYNTVATL (SEQ ID No. 7) loaded T2 cells in the presence of increasing concentrations of bispecific TCR (bssTCR) molecules. The bispecific TCR/mAb diabody molecules IA_3 and ID_1 induced markedly higher lysis of target cells than three alternative dual specificity TCR/mAb molecules. As shown on the right hand sided graph none of the tested bispecific TCR/mAb constructs induced detectable lysis of T2 cells loaded with irrelevant peptide (SEQ ID No. 49).

FIG. 11 shows the results of a LDH-release assay with the bispecific TCR/mAb diabody construct IA_5 targeting tumor-associated peptide PRAME-004 (SEQ ID No. 49) presented on HLA-A*02. CD8-positive T cells isolated from a healthy donor were co-incubated with cancer cell lines UACC-257, SW982 and U2OS presenting differing amounts of PRAME-004:HLA-A*02-1 complexes on the cell surface (approx. 1100, approx. 770 and approx. 240 copies per cell, respectively, as determined by M/S analysis) at an effector:target ratio of 5:1 in the presence of increasing concentrations of TCR/mAb diabody molecules. After 48 hours of co-culture target cell lysis was quantified utilizing LDH-release assays according to the manufacturer's instructions (Promega).

FIG. 12 shows the results of a LDH-release assay with the bispecific TCR/mAb diabody constructs IA_5 and IA_6 utilizing a stability/affinity maturated TCR and an enhanced version thereof, respectively, against the tumor-associated peptide PRAME-004 (SEQ ID No. 49) presented on HLA-A*02. CD8-positive T cells isolated from a healthy donor were co-incubated with the cancer cell line U20S presenting approx. 240 copies per cell of PRAME-004:HLA-A*02-1 complexes or non-loaded T2 cells (effector:target ratio of 5:1) in the presence of increasing concentrations of TCR/mAb diabody molecules. After 48 hours of coculture target cell lysis was quantified utilizing LDH-release assays according to the manufacturer's instructions (Promega).

FIG. 13 shows the results of a heat-stress stability study of the TCR/mAb diabody constructs IA_5 and IA_6 utilizing a stability/affinity maturated TCR and an enhanced version thereof, respectively, against the tumor-associated peptide PRAME-004 (SEQ ID No. 49) presented on HLA-A*02. For this, the proteins were formulated in PBS at a concentration of 1 mg/ml and subsequently stored at 40° C. for two weeks. Protein integrity and recovery was assessed utilizing HPLC-SEC. Thereby the amount of high-molecular weight species was determined according to percentage of peak area eluting before the main peak. Recovery of monomeric protein was calculated by comparing main peak areas of unstressed and stressed samples.

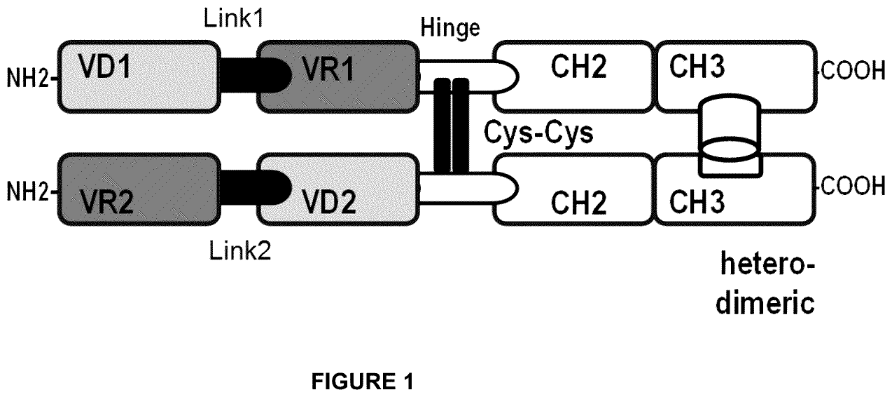

### EXAMPLES

### Example 1

### Design of Fc-Containing Bispecific TCR/mAb Diabodies and Control Molecules

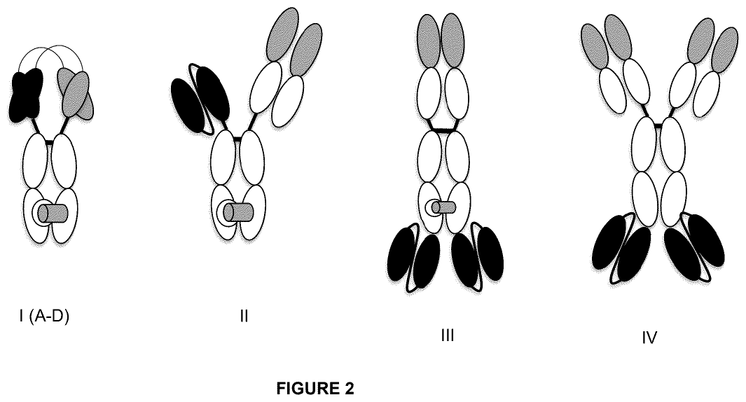

Fc-containing bispecific TCR/mAb diabodies and control molecules (as depicted in FIG. 2) were designed to specifically bind to the human TCR-CD3 complex and to the peptide: MHC complex comprising the HIV-derived peptide SLYNTVATL (SQ ID No. 7) bound to HLA-A2*01. For targeting TCR-CD3 complex, VH and VL domains derived from the CD3-specific, humanized antibody hUCHT1 (V9) described by Zhu et al. (Identification of heavy chain residues in a humanized anti-CD3 antibody important for efficient antigen binding and T cell activation. J Immunol, 1995, 155, 1903-1910) or VH and VL domains derived from the alpha/beta TCR-specific antibody BMA031 described in Shearman et al. (Construction, expression and characterization of humanized antibodies directed against the human alpha/beta T cell receptor. J Immunol, 1991, 147, 4366-73) and employed in the humanized version variant 10 (data generated in-house) were used. For targeting peptide: MHC complex, Valpha and Vbeta domains of the previously described stability and affinity maturated, human single chain T-cell receptor 868Z11 disclosed by Aggen et al. (Identification and engineering of human variable regions that allow expression of stable single-chain T cell receptors. PEDS, 2011, 24, 361-372) were utilized.

In case of Fc-containing bispecific TCR/mAb diabodies DNA-sequences coding for various combinations of VH and VL (corresponding to VD1 and VD2, respectively) and Va and Vb (corresponding to VR1 and VR2, respectively), as well as coding for linkers Link1 and Link2 were obtained by gene synthesis. Resulting DNA-sequences were cloned in frame into expression vectors coding for hinge region, CH2 and CH3 domain derived from human IgG4 [Accession #: K01316] and IgG1 [Accession #: P01857], respectively and were further engineered. Engineered was performed to incorporate knob-into-hole mutations into CH3-domains with and without additional interchain disulfide bond stabilization; to remove an N-glycosylation site in CH2 (e.g. N297Q mutation); to introduce Fc-silencing mutations; to introduce additional disulfide bond stabilization into VL and VH, respectively, according to the methods described by Reiter et al. (Stabilization of the Fv Fragments in Recombinant Immunotoxins by Disulfide Bonds Engineered into Conserved Framework Regions. Biochemistry, 1994, 33, 5451-5459). An overview of produced bispecific TCR/mAb diabodies, the variants as well as the corresponding sequences are listed in Table 1.

TABLE 1
Overview of all generated and evaluated Fc-
containing bispecific TCR/mAb diabodies:
KiH: Knob-into-hole; K/O: Fc-silenced;
KiH-ds: Knob-into-hole stabilized with
artificial disulfide-bond to connect
CH3:CH3′; ds-hUCHT1 (V9): disulfide-
bond stabilized hUCHT1 (V9) variable
domains; Link1: Linker connecting
VR1 and VD1.

| Molecule | TCR | mAb | SEQ | modifi- |
|---|---|---|---|---|
| | | | IDs | cations |
| IA-IgG4 | 868Z11 | hUCHT1(V9) | SEQ ID | IgG4 (KiH) |
| | | | No. 8 | |
| | | | SEQ ID | |
| | | | No. 9 | |
| IA_1 | 868Z11 | hUCHT1(V9) | SEQ ID | IgG1 (K/O, |
| | | | No. 10 | KiH) |
| | | | SEQ ID | |
| | | | No. 11 | |
| IA_2 | 868Z11 | hUCHT1(V9) | SEQ ID | IgG1 (K/O, |
| | | | No. 12 | KiH-ds) |
| | | | SEQ ID | |
| | | | No. 13 | |
| IA_3 | 868Z11 | ds-hUCHT1(V9) | SEQ ID | IgG1 (K/O, |
| | | | No. 14 | KiH-ds) |
| | | | SEQ ID | |
| | | | No. 15 | |
| ID_1 | 868Z11 | ds-hUCHT1(V9) | SEQ ID | IgG1 (K/O, |
| | | | No. 16 | KiH-ds) |
| | | | SEQ ID | |
| | | | No. 17 | |
| IC_4 | 868Z11 | hBMA031(var10) | SEQ ID | IgG1 (K/O, |
| | | | No. 18 | KiH-ds) |
| | | | SEQ ID | |
| | | | No. 19 | |
| IC_5 | 868Z11 | hBMA031(var10) | SEQ ID | IgG1 (K/O, |
| | | | No. 20 | KiH-ds) |
| | | | SEQ ID | extended |
| | | | No. 21 | Link1 |
| ID_4 | 868Z11 | hBMA031(var10) | SEQ ID | IgG1 (K/O, |
| | | | No. 22 | KiH-ds) |
| | | | SEQ ID | |
| | | | No. 23 | |
| ID_5 | 868Z11 | hBMA031(var10) | SEQ ID | IgG1 (K/O, |
| | | | No. 24 | KiH-ds) |
| | | | SEQ ID | extended |
| | | | No. 25 | Link1 |
| IA_5 | R16P1C10I | hUCHT1(Var17) | SEQ ID | IgG1 (K/O, |
| | | | No. 43 | KiH-ds) |
| | | | SEQ ID | |
| | | | No. 44 | |
| IA_6 | R16P1C10I#6 | hUCHT1(Var17) | SEQ_ID | IgG1 (K/O, |
| | | | No. 45 | KiH-ds) |
| | | | SEQ ID | |
| | | | No. 46 | |

Various control molecules exhibiting the same specificities were constructed Table 2 utilizing said VH, VL, Valpha and Vbeta domains in combinations with IgG1- or IgG4-derived constant domains comprising engineered features as described above.

TABLE 2
Overview of all generated and evaluated Fc-
containing bispecific control molecules:
KiH: Knob-into-hole; K/O: Fc-silenced.

| Molecule | TCR | mAb | SEQ IDs | modifications |
|---|---|---|---|---|
| III-IgG4 | 868Z11 | hUCHT1(V9) | SEQ ID No. | IgG4 (KiH) |
| | | | 38 | |
| | | | SEQ ID No. | |
| | | | 39 | |
| IV-IgG4 | 868Z11 | hUCHT1(V9) | SEQ ID No. | IgG4 |
| | | | 40 | |
| | | | SEQ ID No. | |
| | | | 41 | |
| II | 868Z11 | hUCHT1(V9) | SEQ ID No. | IgG1 |
| | | | 33 | (K/O, KiH) |
| | | | SEQ ID No. | |
| | | | 34 | |
| III | 868Z11 | hUCHT1(V9) | SEQ ID No. | IgG1 |
| | | | 35 | (K/O, KiH) |
| | | | SEQ ID No. | |
| | | | 36 | |
| IV | 868Z11 | hUCHT1(V9) | SEQ ID No. | IgG1 (K/O) |
| | | | 37 | |
| | | | SEQ ID No. | |
| | | | 42 | |

### Example 2

### Production and Purification of Fc-Containing Bispecific TCR/mAb Diabodies

Vectors for the expression of recombinant proteins were designed as mono-cistronic, controlled by HCMV-derived promoter elements, pUC19-derivatives. Plasmid DNA was amplified in E. coli according to standard culture methods and subsequently purified using commercial-available kits (Macherey & Nagel). Purified plasmid DNA was used for transient transfection of CHO-S cells according to instructions of the manufacturer (ExpiCHO™ system; Thermo Fisher Scientific). Transfected CHO-cells were cultured for 6-14 days at 32° C. to 37° C. and received one to two feeds of ExpiCHO™ Feed solution.

Conditioned cell supernatant was harvested by centrifugation (4000×g; 30 minutes) and cleared by filtration (0.22 μm). Bispecific molecules were purified using an Äkta Pure 25 L FPLC system (GE Lifesciences) equipped to perform affinity and size-exclusion chromatography in line. Affinity chromatography was performed on protein A columns (GE Lifesciences) following standard affinity chromatographic protocols. Size exclusion chromatography was performed directly after elution (pH 2.8) from the affinity column to obtain highly pure monomeric protein using Superdex 200 μg 16/600 columns (GE Lifesciences) following standard protocols. Protein concentrations were determined on a NanoDrop system (Thermo Scientific) using calculated extinction coefficients according to predicted protein sequences. Concentration, if needed, and buffer exchange was performed using Vivaspin devices (Sartorius). Finally, purified molecules were stored in phosphate-buffered saline at concentrations of about 1 mg/ml at temperatures of 2-8° C.

As therapeutic proteins shall exhibit reasonable stability upon acidic exposure to facilitate robust industrial purification processes the percentage of monomeric protein eluting from the protein A capture column was assessed (Table 3). It is obvious that the introduction of stabilizing mutations into molecules as well as selection of distinct orientations of binding domains markedly impact the stability upon acidic exposure.

TABLE 3
Fraction of monomeric protein after
acidic elution from capture column:

| | Monomer eluted from |
|---|---|
| | capture column |
| Molecule | (% of total peak area) |
| IA-IgG4 (VH-beta) | n.d. |
| IA_1 (VH-beta) | 49 |
| IA_2 (VH-beta) | 54 |
| IA_3 (dsVH-beta) | 63 |
| ID_1 (alpha-dsVH) | 46 |
| IC_4 (VH-alpha) | 62 |
| IC_5 (VH-alpha) | 67 |
| ID_4 (alpha-VH) | 65 |
| ID_5 (alpha-VH) | 69 |
| II | 39 |
| III | 51 |
| IV | 76 |

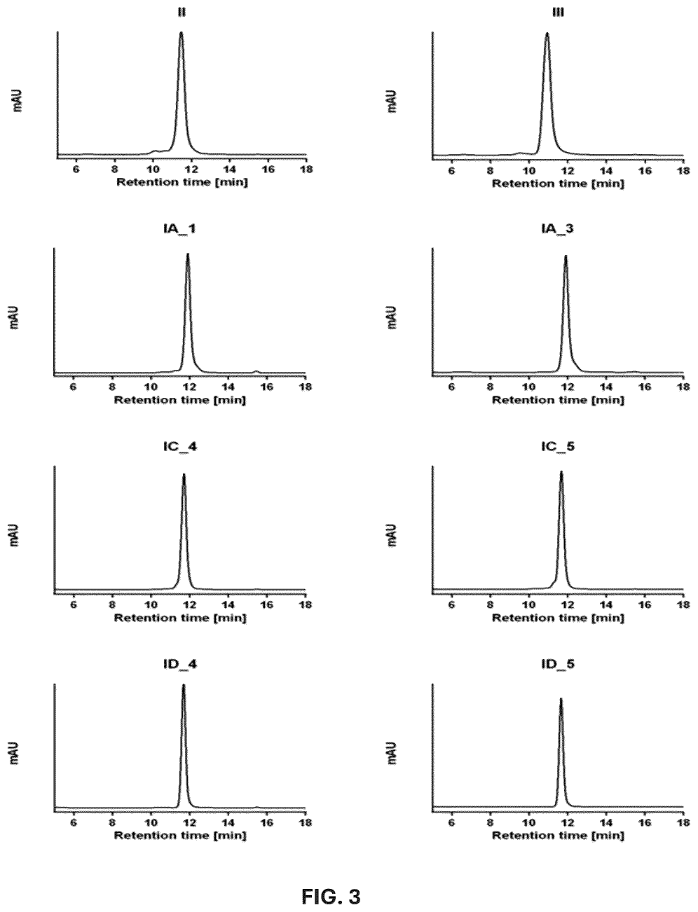

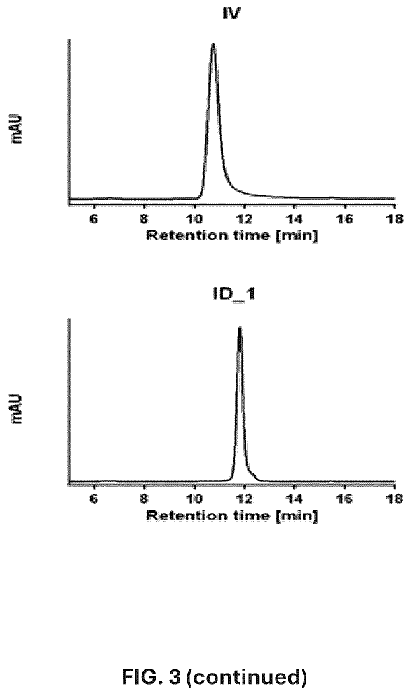

After size exclusion chromatography, the purified bispecific molecules demonstrated high purity (>93% of monomeric protein) as determined by HPLC-SEC on MabPac SEC-1 columns (5 μm, 7.8×300 mm) running in 50 mM sodium-phosphate pH 6.8 containing 300 mM NaCl within an Agilent 1100 system (see FIG. 3). Non-reducing and reducing SDS-PAGE confirmed the purity and expected size of the different dual specificity TCR/mAb molecules (data not shown).

### Example 3

### Specific and Target Cell-Dependent T Cell Activation Induced by Fc-Containing TCR/mAb Diabodies

The potency of Fc-containing TCR/mAb diabodies with respect to T cell activation was assessed using the T Cell Activation Bioassay (Promega). The assay consists of a genetically engineered Jurkat cell line that expresses a luciferase reporter driven by an NFAT-response element (NFAT-RE). Assays were performed according to the manufacturer. Briefly, T2 cells either loaded with the HIV-specific peptide SLYNTVATL (SEQ ID No. 7) or left without peptide loading (unloaded control) were subsequently co-cultured with Promega's modified Jurkat cells in presence of increasing concentrations of bispecific TCR/mAb molecules. Jurkat reporter T cell activation was analyzed after 16-20 hours by measuring luminescence intensity.

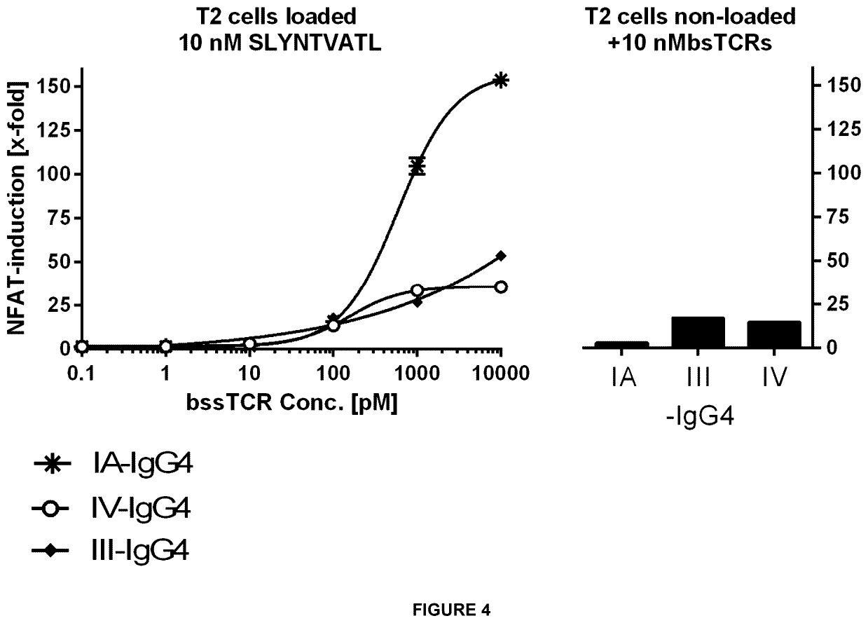

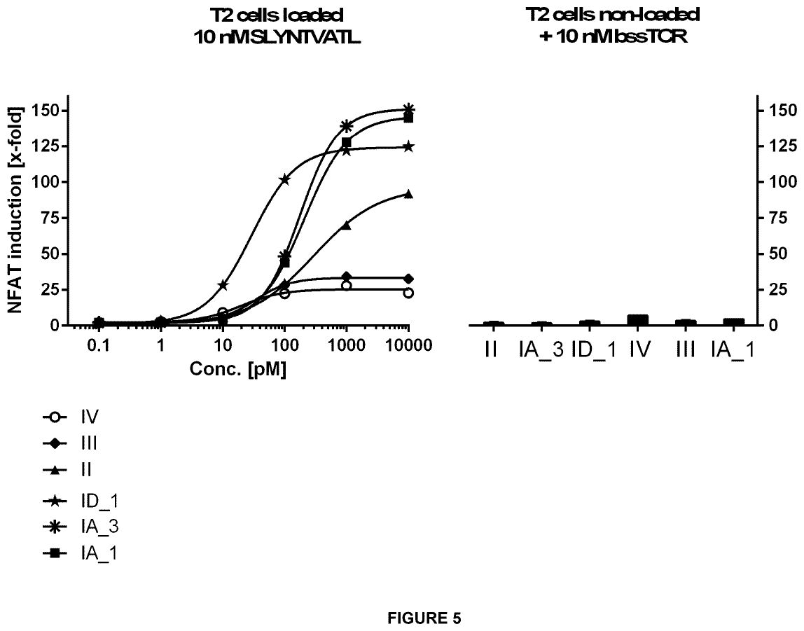

Representative potency assay results are depicted for IgG4-based (FIG. 4) and IgG1-based bispecific TCR/mAb molecules (FIG. 5), respectively. The data indicate that regardless of the IgG isotype of the constant domains used, the Fc-containing TCR/mAb diabody constructs IA and ID showed superior T cell activation compared to the alternative bispecific TCR/mAb constructs II, III and IV as measured by the magnitude of activation and/or respective EC50-values. Furthermore, the unspecific T cell activation of Fc-containing TCR/mAb diabodies induced against unloaded T2 cells was reduced or at least equal to the level of unspecific activation observed for the alternative bispecific TCR/mAb constructs. According to above results the dual specificity TCR/mAb diabody molecules are preferred molecules for therapeutic intervention as they induce strong effector T cell activation in a highly target-dependent manner.

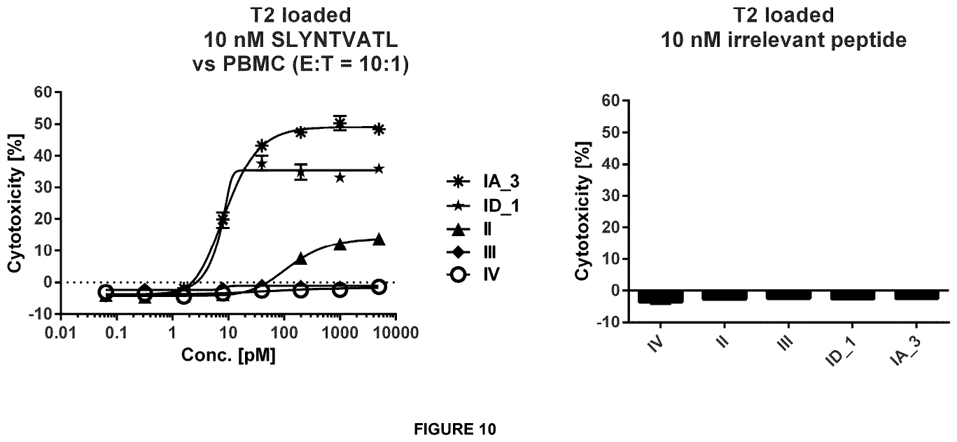

Furthermore LDH-release assay (Promega) was used to quantify the PBMC-mediated lysis of SLYNTVATL (SEQ ID No. 7) peptide-loaded T2 cells induced by the different bispecific TCR/mAb molecules (FIG. 10). In line with the above results of the T Cell Activation Bioassay, again the Fc-containing TCR/mAb diabody constructs IA and ID were superior over the alternative bispecific TCR/mAb constructs II, III and IV as indicated by the increased absolute level of target cell lysis and the lower TCR bispecific concentration needed to achieve half-maximal (EC50) killing of target cells. As for TCR/mAb constructs II, III and IV, the TCR/mAb diabody constructs IA and ID did not induce lysis of T2 cells loaded with irrelevant peptide (SEQ ID No. 49), proving the target-specific lysis to the T2 cells.

### Example 4

### Development of Fc-Containing Bispecific TCR/mAb Diabodies as a Molecular Platform

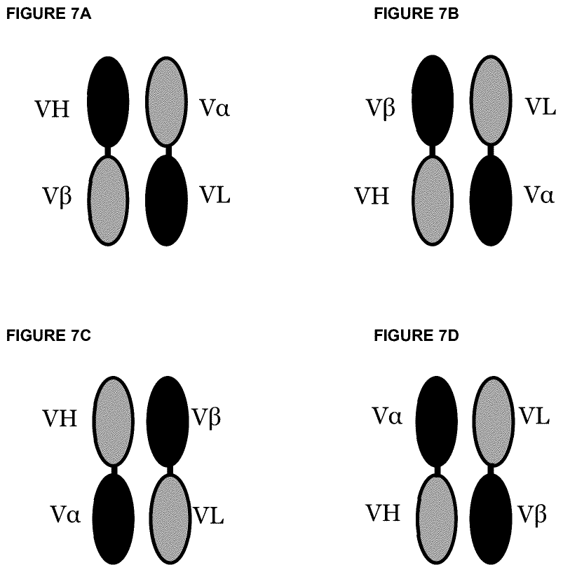

Fc-containing bispecific TCR/mAb diabody constructs were designed to serve as molecular platform to provide the scaffold for different TCR-derived and mAb-derived variable domains targeting different peptide: MHC complexes and effector cell surface antigens, respectively. To validate the suitability as platform, the mAb-derived variable domains were exchanged in a first set of molecules. The variable domains of hUCHT1 (V9) anti-CD3 antibody (construct ID_1) were replaced against the domains of the hBMA031 (var10) anti-TCR antibody employing the same domain orientation (constructs ID_4 and ID_5) or a different orientation (IC_4, IC_5) (see Table 1 and FIG. 7 for details). Expression, purification and characterization of these molecules were performed as described above. Purity and integrity of final preparations exceeded 92% according to HPLC-SEC analyses.

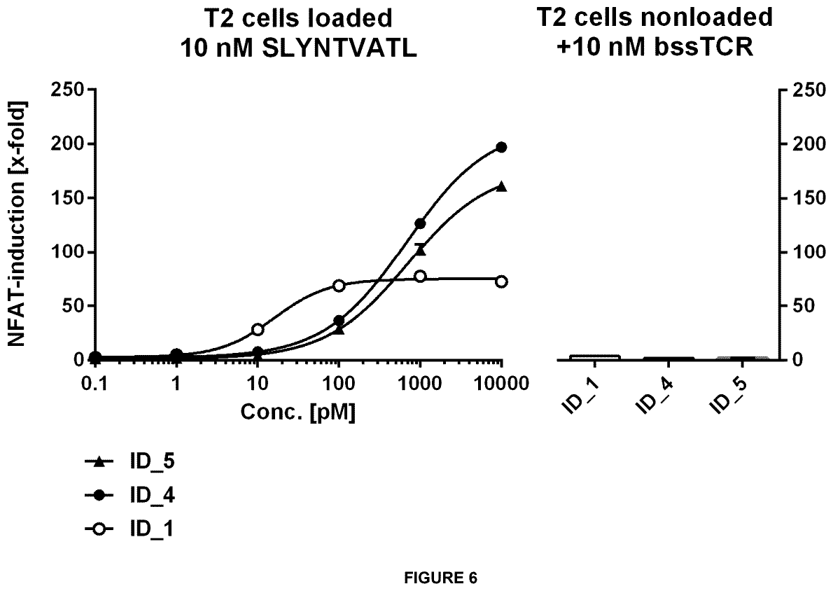

The potency assay results revealed target-dependent Jurkat reporter T cell activation and minimal unspecific activity against unloaded T2 cells for both antibody variable domains hUCHT1 (construct ID_1) and hBMA031 (constructs ID_4 and ID_5) supporting the platform suitability of the dual specificity TCR/mAb diabody constructs (FIG. 6). Notably, when the variable TCR and mAb domains of the constructs ID_4 and ID_5 were switched on each polypeptide chain resulting in constructs IC_4 and IC_5 no T cell activation was observed (data not shown). The latter finding indicate that despite bispecific TCR/mAb diabodies can be used as platform construct for incorporating different TCR and mAb variable domains a thorough optimization of the domain orientation is required to achieve optimal activity of the molecules.

### Example 5

### Stability of Fc-Containing Bispecific TCR/mAb Diabodies

Stability of the bispecific TCR/mAb molecules was initially assessed utilizing the Protein Thermal Shift Assay (Thermo Fisher Scientific) according to the instructions of the manufacturer using a 7500 Real time PCR system (Applied Biosciences). Briefly, purified molecules were mixed with PTS buffer and PTS dye and subjected to a raising temperature gradient constantly monitoring fluorescence of samples. Recorded fluorescence signals were analyzed using PTS software (Thermo Fisher Scientific) and melting temperatures (T_{M}) were calculated by the derivative method.

Stressed stability studies were conducted by storage of purified molecules dissolved in PBS at 40° C. for up to two weeks. Samples were analyzed with regard to protein integrity using HPLC-SEC and potency using the T Cell Activation Assay (Promega) as described above.

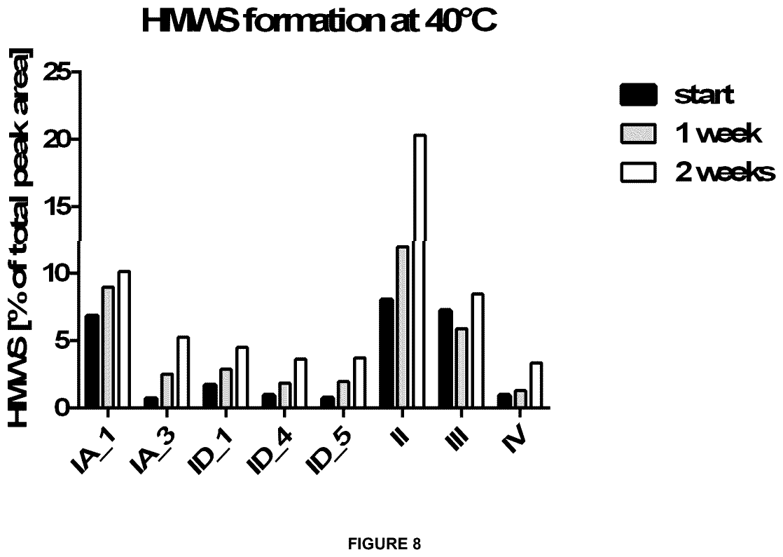

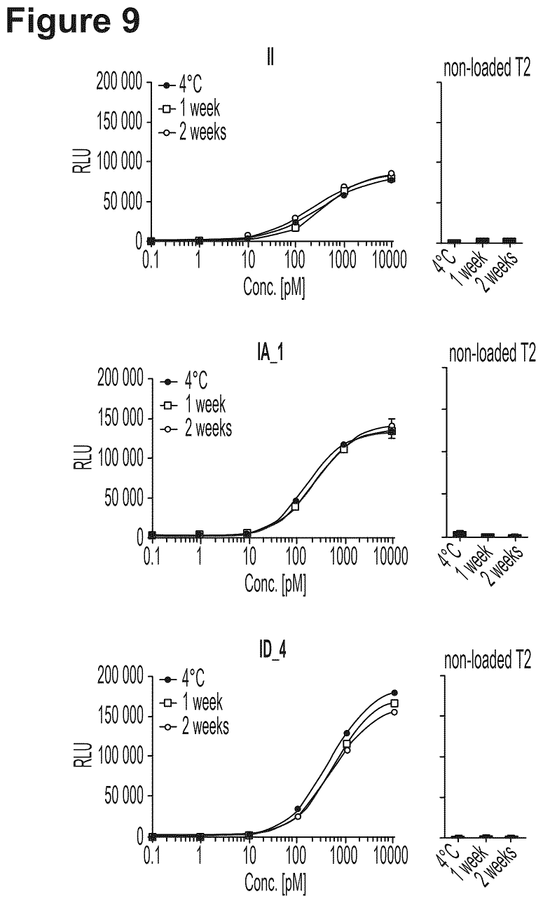

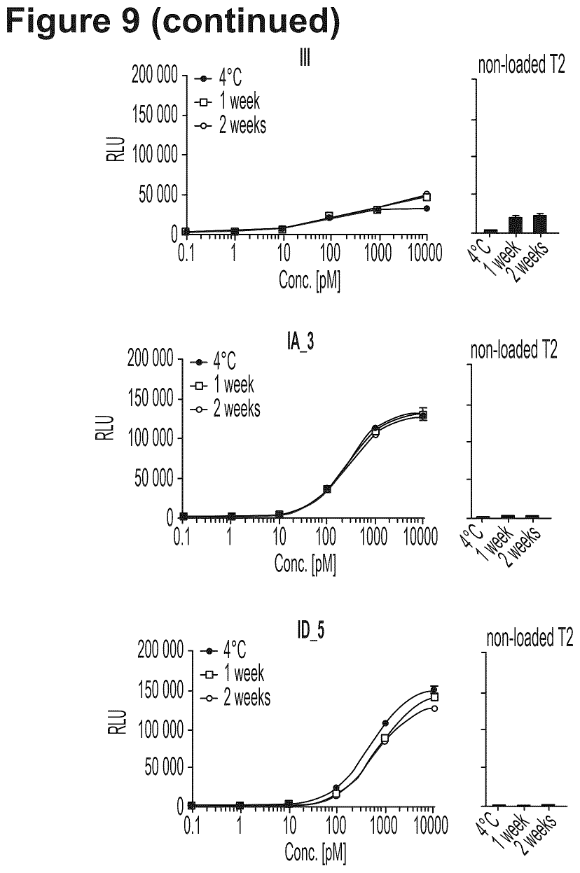

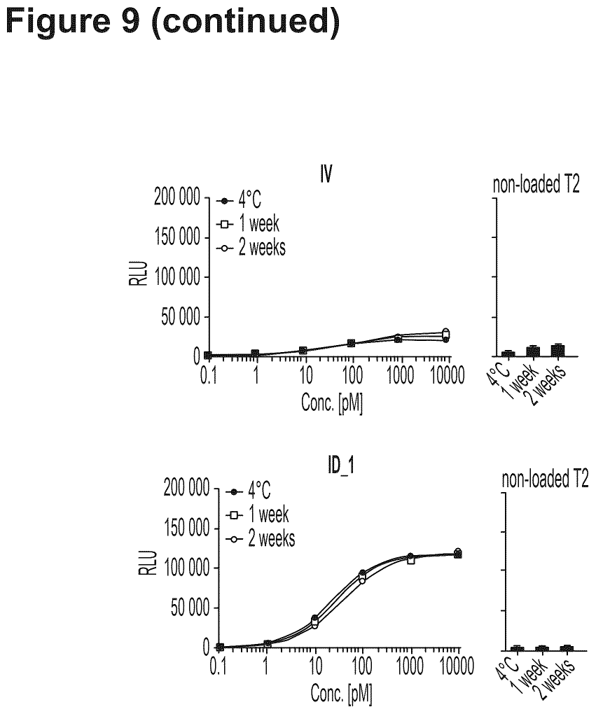

As expected storage at 40° C. induced the formation of aggregates/high-molecular weight species as determined by HPLC-SEC analyses (see FIG. 8). Results of potency assays of IgG1-based molecules after purification and incubation at 40° C. are shown in FIG. 9. Although neither of the tested molecules did show a significant reduction of potency after storage at 40° C., it was observed that the stressed molecules III and IV induced a significant amount of unspecific (i.e. target-independent) Jurkat T cell activation. In contrast, the bispecific TCR/mAb diabodies retained their target-dependent potency, despite the presence of some aggregates as seen in HPLC-SEC.

### Example 6

### Generation of Cancer-Targeting Bispecific TCR/mAb Diabody Molecules

To further validate the platform capabilities of bispecific TCR/mAb diabody constructs, the TCR-derived variable domains were exchanged with variable domains of a TCR, which was stability/affinity maturated by yeast display according to a method described previously (Smith et al, 2015, T Cell Receptor Engineering and Analysis Using the Yeast Display Platform. Methods Mol Biol. 1319:95-141). The TCR variable domains specifically binding to HIV-derived peptide SLYNTVATL (SEQ ID No. 7) in the context HLA-A*02 were exchanged with TCR variable domains specifically binding to the tumor-associated peptide PRAME-004 (SEQ ID No. 49) bound to HLA-A*02. Furthermore, the variable domains of the humanized T-cell recruiting antibody hUCHT1 (V9) were exchanged against variable domains of hUCHT1 (Var17), a newly humanized version of the UCHT1 antibody, resulting in the PRAME-004-targeting TCR/mAb diabody molecule IA_5 (comprising SEQ ID No. 43 and SEQ ID No. 44). Expression, purification and characterization of this molecule was performed as described in Example 2. Purity and integrity of final preparation exceeded 96% according to HPLC-SEC analysis.

Binding affinities of bispecific TCR/mAb diabody constructs towards PRAME-004:HLA-A*02 were determined by biolayer interferometry. Measurements were done on an Octet RED384 system using settings recommended by the manufacturer. Briefly, purified bispecific TCR/mAb diabody molecules were loaded onto biosensors (AHC) prior to analyzing serial dilutions of HLA-A*02/PRAME-004.

The activity of this PRAME-004-targeting TCR/mAb diabody construct with respect to the induction of tumor cell lysis was evaluated by assessing human CD8-positive T cell-mediated lysis of the human cancer cell lines UACC-257, SW982 and U2OS presenting different copy numbers of PRAME-004 peptide in the context of HLA-A*02 on the tumor cell surface (UACC-257-about 1100, SW982-about 770, U2OS-about 240 PRAME-004 copies per cell, as determined by quantitative M/S analysis) as determined by LDH-release assay.

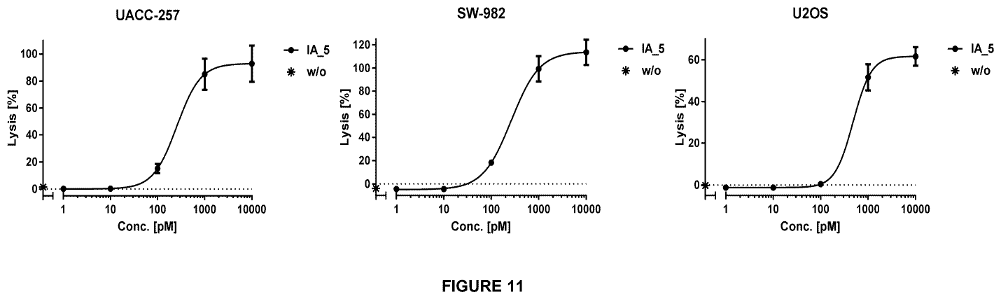

As depicted in FIG. 11, the PRAME-004-targeting TCR/mAb diabody construct IA_5 induced a concentration-dependent lysis of PRAME-004 positive tumor cell lines. Even tumor cells U2OS expressing as little as 240 PRAME-004 copy numbers per tumor cell were efficiently lysed by this TCR/mAb diabody molecule. These results further demonstrate that TCR/mAb diabody format is applicable as molecular platform allowing to introduce variable domains of different TCRs as well as variable domains of different T cell recruiting antibodies.

### Example 7

### Engineerability of TCR/mAb Diabody Constructs

The variable TCR domains utilized in construct IA_5 were further enhanced regarding affinity towards PRAME-004 and TCR stability, and used for engineering into TCR/mAb diabody scaffold resulting in construct IA_6 (comprising SEQ ID No. 45 and SEQ ID No. 46). Expression, purification and characterization of TCR/mAb diabody molecules IA_5 and IA_6 were performed as described in example 2. Purity and integrity of final preparations exceeded 97% according to HPLC-SEC analysis.

Potency of the stability and affinity enhanced TCR/mAb diabody variant IA_6 against PRAME-004 was assessed in cytotoxicity experiments with the tumor cell line U2OS presenting low amounts of PRAME-004:HLA-A*02 or non-loaded T2 cells as target cells and human CD8-positive T cells as effector cells.

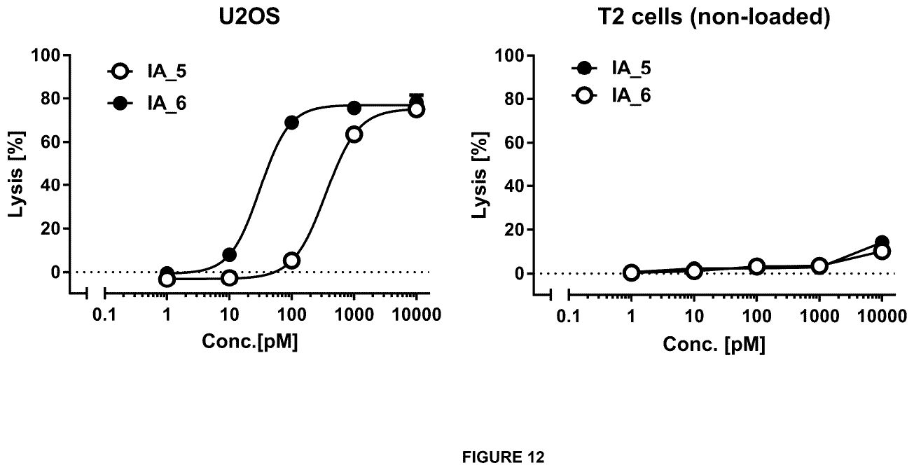

As depicted in FIG. 12, the inventors observed and increased cytotoxic potency of the TCR/Ab diabody molecule IA_6 comprising the variable domains of the stability/affinity enhanced TCR variant when compared to the precursor construct IA_5. For both constructs, IA_5 and IA_6, the PRAME-004-dependent lysis could be confirmed as no cytolysis of target-negative T2 cells was detected.

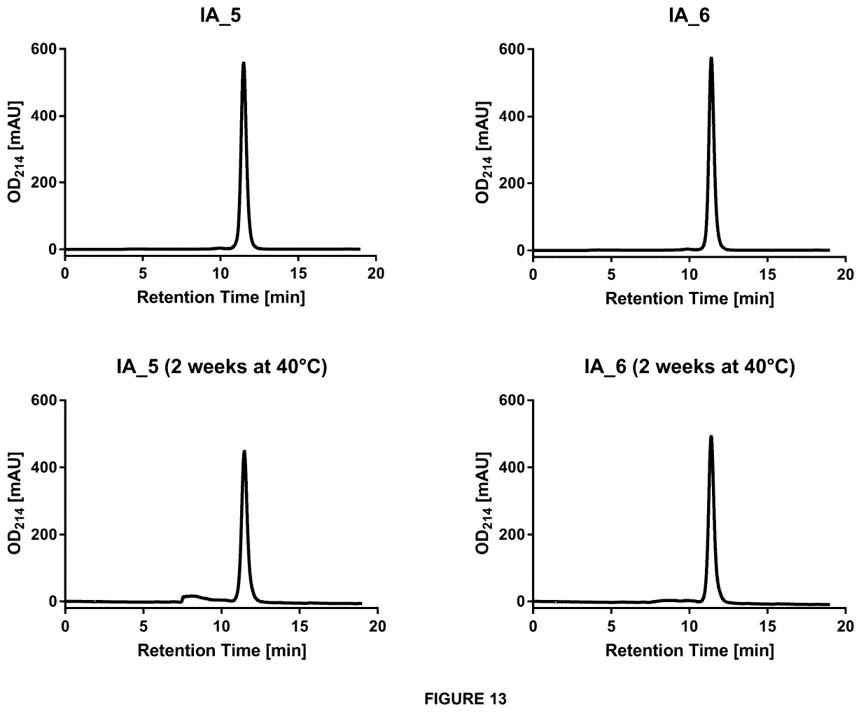

The protein construct were further subjected to heat-stress at 40° C. for up to two weeks to analyze stability of the PRAME-004-specific TCR/mAb diabody variants IA_5 and IA_6. HPLC-SEC analyses after heat-stress revealed a significantly improved stability of the variant IA_6 when compared to the precursor construct IA_5 (see FIG. 13). The temperature-induced increase of high-molecular species (i.e. eluting before the main peak) of the constructs was less pronounced for IA_6 than for IA_5. In line with this result, the recovery of intact, monomeric protein after heat-stress was 87% and 92% for IA_5 and IA_6, respectively.

These exemplary engineering data demonstrate that the highly potent and stable of TCR/mAB diabody constructs can further be improved by incorporating stability/affinity enhanced TCR variable domains resulting in therapeutic proteins with superior characteristics.

### Example 8

### Examples for Preferred Constructs

In addition to the HIV-specific TCR bispecific construct as described herein (Seq ID No. 16 and Seq ID No. 17, in orientation D), the invention further provides several other exemplary HIV-specific constructs that were tested. These constructs are based on an improved humanized variants of the underlying antibody against CD3 (UCHT1) that were fused with the HIV-specific TCR 868 as disclosed herein in all four possible orientations (Seq ID No. 51 to Seq ID No. 58, in orientations A-D).

The humanization of UCHT1 was performed using VH-1-46 and VK1-018 as acceptor frameworks for the heavy and light chain CDRs, respectively. J-segments selected were JK1 and JH4, for light and heavy chain, respectively.

The results as obtained are shown in the following Table 4:

| | V9 (Zhu | Present |
|---|---|---|
| | et al, 1995) | invention |
| DRB1 score | 1232 | ~1190 |
| Titre [mg/L] | 0.75 | 3 |
| Tm of F(ab) [° C.] | 83.0 | 86.4 |
| EC50 of effector cell | 63 | 8 |
| activation [pM] | | |

The data in table 4 shows that the inventive humanization is potentially less immunogenic (lower DRB1-score); the molecules are more stable (increase in melting temperature of about 3° C.); and more potent (˜8× decreased EC50), compared with the standard (V9) (for assay, see example 3).

What is claimed is:

1. A dual specificity polypeptide molecule comprising a first polypeptide chain and a second polypeptide chain, wherein:
a) the first polypeptide chain comprises
a1) a first variable domain (VD1) of an antibody,
a2) a first variable domain (VR1) of a T cell receptor (TCR), and
a3) a first linker (LINK1) connecting said domains;
b) the second polypeptide chain comprises
b1) a second variable domain (VR2) of a TCR,
b2) a second variable domain (VD2) of an antibody, and
b3) a second linker (LINK2) connecting said domains;
wherein said first variable domain (VD1) and said second variable domain (VD2) associate to form a first binding site (VD1) (VD2) that specifically binds a cell surface antigen of a human immune effector cell;
wherein the first variable domain (VR1) is one of a TCR Vα domain and a TCR Vβ domain, and the second variable domain (VR2) is the other one of the TCR Vα domain and the TCR Vβ domain, said first variable domain (VR1) and said second variable domain (VR2) associate to form a second binding site (VR1) (VR2) that specifically binds an MHC-associated peptide epitope;
wherein said two polypeptide chains are fused to human IgG hinge domains and/or human IgG Fc domains or dimerizing portions thereof;
wherein said two polypeptide chains are connected by covalent and/or non-covalent bonds between said hinge domains and/or Fc-domains;
wherein said dual specificity polypeptide molecule is capable of simultaneously binding the cell surface molecule and the MHC-associated peptide epitope; and
wherein the order of the variable domains in the two polypeptide chains is selected from VD1-VR1 and VR2-VD2 or VD2-VR2 and VR1-VD1,
wherein
(i) first and second variable domains of an antibody VD1 and VD2 comprise variable domains of the alpha/beta TCR-specific antibody hBMA031 or wherein the first and second variable domains of an antibody VD1 and VD2 comprise amino acid sequences having at least 90% identity to the variable domains of the alpha/beta TCR-specific antibody BMA031; or
(ii) the first and second variable domains of an antibody VD1 and VD2 comprise the variable domains of the humanized T-cell recruiting antibody hUCHT1 or wherein the first and second variable domains of an antibody VD1 and VD2 comprise amino acid sequences having at least 70% identity to the variable domains of the humanized T-cell recruiting antibody hUCHT1.

2. A nucleic acid or nucleic acids encoding for the first polypeptide chain and the second polypeptide chain according to claim 1.

3. An expression vector or expression vectors comprising the nucleic acid or the nucleic acids according to claim 2.

4. A host cell comprising the expression vector or the expression vectors according to claim 3.

5. A pharmaceutical composition comprising the dual specificity polypeptide molecule according to claim 1 and one or more pharmaceutically acceptable carriers or excipients.

6. A pharmaceutical composition comprising the nucleic acids or the nucleic acids according to claim 2 and one or more pharmaceutically acceptable carriers or excipients.

7. A pharmaceutical composition comprising the expression vector or the expression vectors according to claim 3 and one or more pharmaceutically acceptable carriers or excipients.

8. A pharmaceutical composition comprising the host cell according to claim 4 and one or more pharmaceutically acceptable carriers or excipients.

9. A method for the prevention or the treatment of a disease or disorder comprising administering a therapeutically effective amount of the dual specificity polypeptide molecule according to claim 1 to a patient in need thereof, wherein the disease or the disorder is selected from cancer, infectious diseases, and immunological disorders.

10. A method for the prevention or the treatment of a disease or disorder comprising administering a therapeutically effective amount of the nucleic acid or the nucleic acids according to claim 2 to a patient in need thereof, wherein the disease or the disorder is selected from cancer, infectious diseases, and immunological disorders.

11. A method for the prevention or the treatment of a disease or disorder comprising administering a therapeutically effective amount of the expression vector or the expression vectors according to claim 3 to a patient in need thereof, wherein the disease or the disorder is selected from cancer, infectious diseases, and immunological disorders.

12. A method for the prevention or the treatment of a disease or disorder comprising administering a therapeutically effective amount of the host cell according to claim 4 to a patient in need thereof, wherein the disease or the disorder is selected from cancer, infectious diseases, and immunological disorders.

13. The dual specificity polypeptide molecule of claim 1, wherein the order of the variable domains in the two polypeptide chains is VD1-VR1 and VR2-VD2.

14. The dual specificity polypeptide molecule of claim 1, wherein the order of the variable domains in the two polypeptide chains is VD2-VR2 and VR1-VD1.

15. The dual specificity polypeptide molecule of claim 1, wherein the first and second variable domains of an antibody VD1 and VD2 comprise amino acid sequences having at least 95% identity to the variable domains of the alpha/beta TCR-specific antibody BMA031.

16. The dual specificity polypeptide molecule of claim 1, wherein the first and second variable domains of an antibody VD1 and VD2 comprise amino acid sequences having at least 98% identity to the variable domains of the alpha/beta TCR-specific antibody BMA031.

17. The dual specificity polypeptide molecule of claim 1, wherein the variable domains of the alpha/beta TCR-specific antibody BMA031 comprise a heavy chain variable domain (VH) comprising the amino acid sequence of the VH as comprised in SEQ ID NO: 23 and a light chain variable domain (VL) comprising the amino acid sequence of the VL as comprised in SEQ ID NO: 22.

18. The dual specificity polypeptide molecule of claim 1, wherein the first and second variable domains of an antibody VD1 and VD2 comprise amino acid sequences having at least 80% identity to the variable domains of the humanized T-cell recruiting antibody hUCHT1.

19. The dual specificity polypeptide molecule of claim 1, wherein the first and second variable domains of an antibody VD1 and VD2 comprise amino acid sequences having at least 90% identity to the variable domains of the humanized T-cell recruiting antibody hUCHT1.

20. The dual specificity polypeptide molecule of claim 1, wherein the variable domains of the humanized T-cell recruiting antibody hUCHT1 comprise a VH comprising the amino acid sequence of the VH as comprised in SEQ ID NO: 44 and a VL comprising the amino acid sequence of the VL as comprised in SEQ ID NO: 43.
